(19) United States
(12) Patent Application Publication
(10) Pub. No.: US 2010/0198274 A1
(43) Pub. Date: Aug. 5, 2010

(54) Intervertebral disc inserting device

(76) Inventors: Jeffrey Eric Yeung, San Jose, CA (US); Teresa T. Yeung, San Jose, CA (US)

Correspondence Address: Jeffrey E. Yeung, 834 North White Road, San Jose, CA, 95127, US

(21) Appl. No.: 12/798,773

(22) Filed: Apr. 10, 2010

Related U.S. Application Data
(63) Continuation of application No. 10/470,181, filed on Jul. 21, 2003.

(51) Int. Cl. A61B 17/56

(52) U.S. Cl. 606/86 A

(57) ABSTRACT

With limited nutrients within the avascular disc, the water-retaining proteoglycans begin to diminish, resulting in dehydration, flattening and/or bulging of the disc. The flattened disc causes segmental instability, eroding the facet joints and causing pain.

In this invention, a disc inserting device contains a horizontally oriented protrusion with superior and inferior plateaus for inserting into the degenerated disc to maintain or restore disc height. The horizontally oriented protrusion is adjoined to a vertically oriented concave bracket with screw holes for fastening the concave bracket to the vertebral bodies sandwiching the degenerated disc. Thereby, the disc height is restored and fortified to reduce segmental instability and erosion of facet joints for pain relief. Furthermore, by altering the slopes of the plateaus, thickness and depth of the protrusion, spinal stenosis, scoliosis, kyphosis, lordosis or spondylolisthesis can be corrected with the disc inserting device.

### CROSS REFERENCES

This application is a continuation of U.S. Ser. No. 10/470,181, filed on Jul. 21, 2003 as the US national application of PCT/US2002/04301, filed on Feb. 13, 2002.

This continuation application also claims priority of U.S. Provisional 60/268,666 filed on Feb. 13, 2001; U.S. Provisional 60/297,556 filed on Jun. 11, 2001; U.S. Provisional 60/310,131 filed on Aug. 3, 2001; U.S. Provisional 60/325,111 filed on Sep. 26, 2001; U.S. Provisional 60/330,260 filed on Oct. 17, 2001.

### FIELD OF INVENTION

This invention relates to devices and methods for occupying and maintaining the intervertebral disc space by fastening the disc space inserting devices to one or more vertebral body to treat spine pain.

### BACKGROUND, EXISTING SURGICAL PRACTICES AND PRIOR INVENTIONS

Low-back pain is one of the most prevalent, costly and debilitating ailments afflicting mankind. Seventy to eighty-five percent of all people have back pain at some time in their life. Symptoms are most common among middle-aged adults and are equally common among both men and women. Back pain related to disc disorders, however, is more prevalent among men. The recurrence rate of low back pain ranges from 20% to 44% annually, with lifetime recurrences of 85% (National Institute of Health Guide, Vol. 26, 16, May 16, 1997).

Low back pain is very costly to patients, our health care system and society. For many, no position can ease their pain or numbness, not even bed rest. It is often the reason for decreased productivity due to loss of work hours, addiction to pain-killing drugs, emotional distress, prolonged hospital stays, loss of independent living, unplanned early retirements and even financial ruin. Each year in the US, about 2% of the work force have back injuries covered by worker's compensation, with about $12 billion spent directly on medical costs in 1994.

### Bulging or Herniated Intervertebral Discs

Most back pain is initiated with a defective or damaged intervertebral disc. The disc is comprised of nucleus pulposus and annulus. The nucleus pulposus is highly gelatinous with a composition of 70-90% water, 25-60% proteoglycan (dry weight) and 10-20% collagen (dry weight). The function of the nucleus pulposus is to sustain prolonged compression during the day and to resiliently re-inflate and reestablish disc height during the night. The pulposus is retained and surrounded by layers of cartilaginous annulus. Together the pulposus and the annulus behave as a resilient cushion. In the erect position, the weight of the body constantly compresses upon a stack of these cushions alternating between a series of vertebrae. During constant compression, the pulposus in each disc also behaves as a water reservoir, which is slowly and constantly being squeezed and drained of its water content through the end plates connected to the vertebrae. As a result, the disc height decreases throughout the day. During bed rest, the weight of the body no longer compresses the disc. Due to the water absorbing nature of the nucleus pulposus, the flow of water then reverses from the vascular vertebrae back into the proteoglycan and collagen. As a result, the disc height is reestablished and ready to provide support for another day.

With aging and degeneration, the viscoelastic property of the nucleus pulposus undergoes a transition from fluid-like to solid-like behavior (J. C. Iatridis et. al., Journal of Orthopaedic Research, 15:318-322, 1997). Under dynamic conditions, the gelatinous nucleus pulposus exhibits predominantly solid-like behavior with values for dynamic modulus ranging from 7 to 20 kPa (J. C. Iatridis et. al., J. Biomechanics, Vol. 30, No. 10, 1005-1013, 1997). As a result, both the resiliency and disc height diminish.

Bulges are most commonly reported at the posterior-lateral regions of the discs. The bulging regions are commonly divided into zones. The posterior region where the spinal cord is located is called the central zone. Adjacent to both sides of the central zone are the entrance zones, followed by pedicle zones, the exit zones, and the far lateral zones. Bulges at the far lateral zones, the most accessible area, have the highest surgical success rate.

Some causes that contribute to low back pain are classified. Type I: Acute back sprain involves damage to ligaments, muscles or even the vertebral end plates from physical overload. Type II: Organic idiopathic spine pain occurs from increased fluid uptake by the disc. Type III: Disruption of posteriolateral annular fibers irritates nerves associated with the sacroiliac region, buttock and the back of the thigh. This situation may resolve itself through reabsorption or neutralization by phagocytosis of the disrupted annular fibers. Type IV: Nerve root irritation by the bulging disc leads to sciatica. This type of disc protrusion is traditionally repaired surgically by tissue removal, chemonucleolysis or percutaneous discectomy. Type V: Nerve irritation by wandering sequestered disc material has unpredictable exacerbation and remission. Type VI: Sequestrum of the annulus and/or nucleus into the spinal canal or intervertebral foramen results in nerve irritation from inflammation, mechanical pressure, chemical irritation, autoimmune response or combinations of irritants. Type VII—A degenerated disc, with substantial decrease in mechanical properties, is often associated with pain and disability.

The most common reason for recurrent pain is the bulging or herniation of an intervertebral disc. The traditional surgical treatment for a bulging or herniated disc is a series of tissue removing, filling and supporting procedures: (1) laminectomy, excision of the posterior arch of a vertebra which covers part of the herniated disc, (2) discectomy, removal of the disc, (3) bone harvesting usually from the patient's iliac crest, (4) donor bone packing into the vacant disc space, (5) supporting adjacent vertebral bodies with rods, connectors, wire and screws, (6) bone cement filling the donor site, and finally (7) closing multiple surgical sites.

Numerous postoperative complications can occur after a back surgery. The major ones are lumbar scarring and vertebral instability. The scar tissue extends and encroaches upon the laminectomy site and intervertebral foramen, then once again, pain returns, which leads to more surgery. In fact, repeat operations are very common, 10-20%. Unfortunately, the success rates of repeat operations are often less, in some cases, far less than the first. More operations lead to more scarring and more pain. Current recommendations to the patients are to avoid surgical procedures unless the pain and inconveniences are absolutely unbearable. Even for the fortunate patients with long term success following discectomies performed twenty years ago, their isokinetic test results clearly indicate weaknesses compared to populations without discectomies.

There was and still is increasing interest in more effective and less invasive surgical techniques on the spine to reduce both trauma and cost. The major objectives of surgery on bulging or herniated lumbar discs are (1) decompression of the involved nerve root or roots, and (2) preservation of bony spine, joints and ligaments.

Chymopapain is an enzyme used to digest the nucleus pulposus, the viscous and gel-like substance in the central portion of the disc, which then creates space for the bulging part of the disc to pull back from the encroached nerve root. The needle for injecting the chymopapain is accurately guided to the mid-portion of the disc by a stereotaxic device. The overall success rate is documented as high as 76%. However, some patients are allergic to the treatment and die from anaphylaxis. Some suffer from serious neuralgic complications, including paraplegia, paresis, cerebral hemorrhage and transverse myelitis.

Percutaneous nuclectomy is an alternative method for removing nucleus pulposus without the allergic reaction of chymopapain, and it rarely causes epidural scarring. Similar to the chymopapain injection, a needle followed by a tube-like instrument is guided and confirmed by anteroposterior and lateral fluoroscopy. The nucleus pulposus is then removed mechanically or by vacuum. As a result, a void is created within the disc and the bulging decreases, like the air being released from a worn out tire, with the hope that the bulging portion of the disc will recede and no longer encroach upon the adjacent nerve root. This type of procedure is often referred to as one of the decompression procedures. However, the amount of nucleus pulposus removed has been documented to be insignificantly small, with unpredictable results and a low rate of success.

Recently, several devices (U.S. Pat. No. 5,800,550 to Sertich, 1998; U.S. Pat. No. 5,683,394 to Rinner, 1997; U.S. Pat. No. 5,423,817 to Lin, 1995; U.S. Pat. No. 5,026,373 to Ray et. al., 1991) were designed to fortify the disc space between vertebrae. These types of devices are frequently referred to as spinal cages. Before inserting the device into the disc, the affected disc with portions of vertebral bone above and below the disc are cored out. Usually two holes are cored on each side of the disc for insertion of two spinal cages. Donor bone or bone growth promoting substances are packed into the porous cages. As the vertebrae heal from the coring, new bone grows into and permanently secures the porous cages. The purpose of using spinal cages is to replace the disc and keep the vertebrae apart. However, these vertebrae are permanently fused to each other, without resilient cushion, rotation or mobility.

An improved version of a metallic spinal fusion implant (U.S. Pat. No. 5,782,832 to Larsen and Shikhman, 1998) tries to provide both rotational and cushioning capabilities. This invention resembles a disc prosthesis following a complete discectomy. Therefore, at the least, all the complications and postsurgical problems associated with a discectomy also apply when this device is used.

Patent application, WO 00/40159 by Yeung et al., introduces some devices and methods for fastening herniated and/or bulging discs. The application covers a resiliently bent fastener, screw, suture, staple and tack, with methods to fasten and hold in the bulging annulus. Another patent application, WO 01/95818, by Yeung, introduces more devices and methods for fastening the intervertebral disc to treat nerve impingement, vertebral instability and spinal stenosis.

### Spinal Stenosis

Disc degeneration has been shown to be the first stage in the aging processes of the spine. As the process develops, the circumferential and radial tears of the annulus become evident, proteoglycan and collagen dehydrates (water content of nucleus pulposus fall from 85% to 70%), resulting in decreased disc height. As the annulus continues to degenerate, the disc bulges and/or flattens, narrowing the central canal. The condition is called spinal stenosis. Spinal stenosis is a progressive and dynamic process. Depending on the amount and location of the stenosis, the symptoms may be restricted to a single isolated root, as in lateral recess stenosis, or may involve multiple levels. A normal lumbar canal has a 12-mm or greater anterior-posterior diameter. However, the nerve root within the small neuroforamen is particularly susceptible to impingement from a lateral bulging disc and is often further aggravated by facet joint erosion or alteration.

Mechanical compression of spinal nerve roots from spinal stenosis has a variety of clinical symptoms, including weakness, reflex alterations, pain and paresthesias. Intermittent neurogenic claudication (limping) has been found in patients with stenosis. Clinical features include low back pain and dysesthesia (sense impairment) spreading diffusely down the posteriolateral parts of the lower extremities, often asymmetrically. Pain is typical and often exacerbated by walking and standing. Symptoms disappear with sitting, recumbency or other changes in posture that reverse the lumbar lordosis (curvature). To distinguish clinically between spinal stenosis and herniated disc, restriction of straight-leg raising is frequently not painful in patients with spinal stenosis, but painful in patients with disc herniation. Spinal stenosis complicated by a herniated disc and spondylosis was noted to occur in 39% of 227 patients with low back pain. Spinal stenosis was the only cause of symptoms in only 8% of patients (M. Camins. et. al., The Lumbar Spine, Raven Pres, NY, 1987, pp. 149).

As the disc space narrows, the settling of the facet joints greatly increases mechanical stress, leading to joint erosion. As the joint erodes, the narrowed space of the neuroforamen diminishes. The nerve root is entrapped and surrounded by the pedicle (the bony extension forming the facet joint) superiorly, the bulging disc inferiorly, the vertebral body osteophytes anteriorly and the hypertrophied degenerative facets posteriorly. Most nerve entrapment occurs in the vicinity of the pedicle. This has been referred to as the hidden zone. The nerve root and ganglion are highly protected and covered by bone. Decompression of the nerve root using current surgical technique requires a significant amount of bone and disc removal, making the procedure very invasive. Nerve root impingement at the extraforaminal zone is usually from ligament, lateral disc herniation or tumor.

Although the majority of lumbar spinal conditions should initially be treated conservatively, certain conditions do require urgent surgical intervention. Significant or progressive weakness of the lower extremity in the form of either footdrop or the inability to toe stand may result in irreversible damage. It is imperative to initiate early diagnostic evaluation followed by prompt surgical treatment.

Decompression laminectomy (excision of the posterior arch of a vertebra) is the standard procedure advocated. The ligamentum flavum is usually left intact to protect the dura, and the facet joints are protected. But in certain instances less aggressive laminotomies (removal of a portion of lamina) may be appropriate with hospitalization 5 to 7 days postoperatively. Ambulation may begin within 24 hours after surgery and often on the same day. Despite the invasiveness of the procedure, mortality rate is low (0.1-0.6%). Other complications include neurologic deficit, temporary in 5%, permanent deficit in 1.3%, cerebrospinal fluid fistulas (leakage) 4.6%, infection 0.5%-8.5%, reoperation 9.8% and increased risk of facet fractures.

A 20-year follow-up study, noted complete relief of preoperative signs and symptoms in 68% of patients. The remaining patients (32%) continue having lumbago (pain in low back and buttocks), intermittent claudication (lameness), motor deficit, sciatica (pain radiating from the back into lower extremity), paraplegia (paralysis of the legs) and/or micturition (the passage of urine).

### Segmental Instability

Instability across the motion segment (vertebral body-disc-vertebral body) can occur as the disc degenerates. Segmental instability resembles an out-of-control car riding on one or more flat tires with deflated and unsupported sidewalls. A flattened intervertebral disc causes excessive movement between vertebral bodies, leading to pain in surrounding ligaments and facet joints. Depletion of nucleus pulposus from the percutaneous nuclectomy procedure can accelerate disc flattening or thinning, leading to segmental instability and/or spinal stenosis. Although it might not be grossly detected radiographically, this instability is most apparent during compressional or rotational movements. Under normal conditions, the spinal motion segment and particularly the neuroforamen can smoothly and symmetrically accommodate rotational motions, as well as flexion and extension, without significant alteration of available space. However, as the disc degenerates, the ligaments buckle, the facet joints mal-align and unstable movement appears during routine vertebral motions. With narrowing of the central canal and neuroforamen, unstable vertebral movements produce irritation, inflammation and pain.

Treatment recommended for segmental instability is mostly rest and drug therapy, including analgesics, anti-inflammatory agents, oral steroids, muscle relaxants and antidepressants.

### Spondylolisthesis

The axial compression force upon the L5-S1 level is between 1500 and 2500 N, bending moment between 15 NM and 25 NM. Due to the curvature of the spine, approximately 20% of the axial compression force is a forward-directed shear force. (Bergmark A., Acta Orthop Scand Suppl: 230-238, 1989). As the shear force works on an aging and degenerating disc, the forward sliding process begins. The shear force intensifies as the L5 moves forward and provides more and more leverage. Finally, the ventral (forward) sliding of L5 in relation to S1, called spondylolisthesis, brings a great deal of pain from many possible nerve impingements, including impingement by the transverse process and ligament.

When slippage is less than 50%, vertebral traction alone can usually reposition the L5-S1 disc without removing the L5-S1 disc. Lumbosacral fusion is followed. However, if the slippage is greater than 50%, additional instrumentation may be required to reposition the L5. During the repositioning process, the L5-S1 disc may not be spared. Lumbosacral fusion is necessary and usually done with pedicle screws and instrumentation in an open surgery.

### Deformities of Spine

Most spine deformities are innate. Surgical correction of these deformities is highly invasive and many require repeat surgeries due to instrumentation fatigue/failure or complications. Scoliosis is a condition involving lateral curves or angular deviations of one or more vertebral segments. Commonly known as humpback, kyphosis is an exaggeration of the posterior convexity of the thoracic vertebral column. Three common causes of kyphosis are (1) absence of T-12 vertebral body, (2) malformation and incomplete segmentation of vertebral body, and (3) indentation of anterior portion of vertebral body from compression. Lordosis is an exaggeration of the posterior concavity of the spine characteristic of the lumbar region. Commonly known as swayback, it indicates extreme anterior curvature of the lumbar spine.

### SUMMARY OF INVENTION

Majority of back pain can be traced to degenerated discs, which are likely caused by occlusion of calcified endplates hindering diffusion of nutrients from the vertebral bodies into the avascular intervertebral disc. With limited nutrients within the avascular disc, the water-retaining proteoglycans begin to diminish, resulting in dehydration, flattening and/or bulging of the disc. The flattened disc causes segmental instability, eroding the facet joints and causing pain.

In this invention, a disc inserting device contains a horizontally oriented protrusion with superior and inferior plateaus for inserting into the degenerated disc to maintain or restore disc height. The horizontally oriented protrusion is adjoined to a vertically oriented concave bracket with screw holes for fastening the concave bracket to the vertebral bodies sandwiching the degenerated disc. Thereby, the disc height is restored and fortified to reduce segmental instability and erosion of facet joints for pain relief. Furthermore, by altering the slopes of the plateaus, thickness and depth of the protrusion, spinal stenosis, scoliosis, kyphosis, lordosis or spondylolisthesis can be corrected with the disc inserting device.

REFERENCE NUMBER

| | |
|---|---|
| 100 | Intervertebral disc |
| 101 | Tightening elements |
| 102 | Nerve |
| 103 | Trocar |
| 104 | Sleeve with windows |
| 105 | End-plate |
| 106 | Slit opening |
| 107 | Strut |
| 108 | Head of sleeve with window |
| 109 | Thread |
| 110 | Hole for screw or bolt |
| 111 | Disc compressor |
| 112 | Indented portion |
| 115 | Epiphysis |
| 116 | Bolt head |
| 117 | Stabilizer lumen |
| 118 | End of lift spring |
| 119 | Annulus contact surface |
| 120 | Hole for bolt |
| 121 | Lift spring |
| 122 | Supporting plate |
| 123 | Spinal cord |
| 124 | Delivery device |
| 125 | Coil spring |
| 126 | Pivoting means |
| 127 | Elastic fastening means |
| 128 | Nucleus pulposus |
| 129 | Facet joint |
| 130 | Tip of the compressor |
| 131 | Delivery capsule |
| 132 | Latch |
| 133 | Socket drive |
| 134 | Stabilizer |
| 135 | Lip of stabilizer |
| 139 | Bracket |
| 140 | Sacrum |
| 142 | Superior articular process |
| 143 | Inferior articular process |
| 159 | Vertebral body |
| 160 | Tissue ingrowth opening |
| 161 | Bolt |
| 162 | Nut |
| 163 | Washer |
| 164 | Indentation |
| 165 | Slit hole for bolt or screw |
| 167 | Anterior longitudinal ligament |
| 170 | Sloped surface |
| 171 | Plateau surface |
| 172 | Pivotal peg or screw |
| 173 | Stop |
| 176 | Widening tool |
| 177 | Clamp grabber |
| 178 | Lock screw of widening tool |
| 179 | Lock wheel of widening tool |
| 180 | Hinge of lock screw |
| 181 | Handle of widening tool |
| 182 | Pivotal joint of widening tool |
| 183 | Lock slot |
| 184 | Impingement of nerve |
| 185 | Trocar guide |
| 187 | Screw |
| 188 | Casing of compressor |
| 194 | Ventral/dorsal ramus nerve root |
| 195 | Posterior longitudinal ligament |
| 196 | Nerve shield |
| 198 | Clamp |
| 199 | Widening mount |
| 201 | Support mount |
| 202 | Trough on shield |
| 212 | Strap |
| 213 | Distal tip of the nerve shield |
| 214 | Open channel of nerve shield |
| 215 | Staple |
| 216 | Sinuvertebral nerve |
| 217 | Screw entry |
| 218 | Biodegradable sleeve |
| 220 | Trocar sleeve |
| 221 | Label showing direction of curved |
| | trocar |
| 223 | Trough or indentation of compressor |
| 224 | Bleeding sites |
| 225 | Lumen of sleeve with window |
| 226 | Screw head |
| 228 | Opening for socket or screw driver |
| 229 | Locking mechanism |
| 230 | Dilator |
| 231 | Indentation of disc clamp |
| 233 | Outer surface |
| 234 | Spinal fusion |

### BRIEF DESCRIPTION OF THE DRAWINGS

FIG. 1 depicts a common disc 100 protrusion at or near the neuroforamen, impinging upon the ventral/dorsal ramus nerve root 194.

FIG. 2 shows a nerve shield 196 with a thin but blunt distal tip 213 to reach into or near the neuroforamen and a trough 202 to protect the nerve exiting from the neuroforamen.

FIG. 3 indicates the nerve shield 196 reaching into or near the neuroforamen by sliding over the bulging annulus of the disc 100.

FIG. 4 depicts two nerve shields 196 protecting the nerves 194 from instrumentation.

FIG. 5 shows an elastic clamp 198 comprising two disc compressors 111 with annular contact surfaces 119, sloped surfaces 170, plateau surfaces 171, stops 173, widening 199 and support 201 mounts.

FIG. 6 shows a clamp-widening tool 176 equipped with clamp grabbers 177 and a locking mechanism capable of slow release.

FIG. 7 depicts widening and placement of the disc clamp 198 by the widening tool 176 around the protruded disc 100.

FIG. 8 shows alleviation of nerve 194 impingement by clamping of the bulging annulus with compressors 111. The size of the clamp/compressors 198/111 is enlarged disproportionately to the disc 100, for clarification.

FIG. 9 indicates the locations of compression by the compressors 111. The important compressions are at area C and I, common locations of disc 100 protrusion.

FIG. 10 indicates an elastic strap 212 threaded through the support mount 201 to support the disc clamp 198. The elastic strap 212 is secured by a staple 215 anchored in the vertebral body 159.

FIG. 11 depicts a coronal view of the clamped disc 100 during initial clamping. The sloped surfaces 170 of the compressors 111 rest on the surface of the annulus.

FIG. 12 shows penetration of the sloped surfaces 170 with time. Further penetration is halted by the stops 173 resting on the sides of the vertebral body 159.

FIG. 13 depicts a coronal view of two unsymmetrical compressors 111 installed on a scoliotic vertebral segment.

FIG. 14 shows correction or straightening of the scoliotic vertebral segment with time, by selectively elevating, wedging or shimming the concave side of the vertebral segment.

FIG. 15 depicts a disc clamp 198 with thick compressors 111 installed on a disc 100 displaying spinal stenosis. The size of the clamp/compressors 198/111 is enlarged disproportionately to the disc 100, for clarification.

FIG. 16 shows penetration of the sloped surfaces 170 and plateau surface 171 with time into the disc 100 to thicken the intervertebral disc 100.

FIG. 17 depicts a coronal view of compressors 111 initially installed around a disc 100 displaying spinal stenosis. Bone spurs have grown around the vertebral body 159.

FIG. 18 shows penetration and shimming of the compressors 111 with time into the disc 100 to elevate disc height. The penetration is halted when the stops 173 rest on the vertebral body 159.

FIG. 19 shows that the compressors 111 can be modular components individually fitted on a disc clamp 198.

FIG. 20 depicts the modular compressor 111 comprising an annulus contact surface 119, sloped surface 170, plateau surface 171, stop 173 and pivotal peg 172 for inserting into the clamp.

FIG. 21 indicates a modular compressor 111 including a casing 188 with anchoring screws 187 and the disc contact portion of the compressor 111.

FIG. 22 depicts a vertical cross-sectional view of a compressor 111 with two stops 173, an outer surface 233, upper and lower plateau surfaces 171, sloped surfaces 170 and annular contact surface 119.

FIG. 23 shows a compressor 111 with no stop and a very round annular contact surface 119.

FIG. 24 depicts a compressor 111 with multiple slopes in the sloped surfaces 170.

FIG. 25 shows a compressor 111 with unsymmetrical sloped surfaces 170.

FIG. 26 depicts a compressor 111 with tissue ingrowth openings 160 on the plateau surfaces 171 to promote annular ingrowth and stability of the compressor 111.

FIG. 27 shows a compressor 111 with non-parallel plateau surfaces 171.

FIG. 28 indicates the clamp 198 width measurement and the reach-in distance to stabilize the fastened clamp 198.

FIG. 29 depicts a typical strain vs. stress profile of nickel-titanium (nitinol) alloy suitable for fabricating into a disc clamp 198.

FIG. 30 indicates a compressor 111 pivotally fastened with a screw 187 to a bracket 139.

FIG. 31 shows a one-piece compressor 111 with a bracket 139.

FIG. 32 depicts the one-piece compressor 111 and bracket 139 fastened by two bolts 161 or screws onto the side of the vertebral body 159, compressing the disc 100.

FIG. 33 shows a coronal view of disc 100 compression by the compressors 111 on brackets 139 fastened with bolts 161 and nuts 162 through the vertebral body 159.

FIG. 34 depicts a bolt 161 with two longitudinal slits 106 cut in series. The bolt 161 is made with elastic material, such as nickel-titanium (nitinol).

FIG. 35 depicts the slits 106 being shimmed open and shaped, forming four elastic and compressible struts 107. The length of the bolt 161 is elastically and resiliently shortened.

FIG. 36 shows a sleeve 104 with a lumen 225 and four windows 114, sized and configured to allow protrusion of the elastic struts 107 of the bolt 161, as shown in FIG. 35.

FIG. 37 indicates the insertion of the bolt 161 with the elastic struts 107 being resiliently compressed and fitted within the sleeve 104 in an out-of-phase position.

FIG. 38 depicts protrusion of the opened struts 107 from the windows 114 by turning the bolt 161 relative to the sleeve 104 from the out-of-phase to an in-phase position.

FIG. 39 indicates a coronal view of a spinal stenosis segment fastened with two compressors/brackets 111/139 by two elastic bolts 161 containing slits 106 in out-of-phase position.

FIG. 40 indicates disc 100 compression and penetration with time by the compressors 111, activated or initiated by turning the elastic bolts 161 to in-phase position with the sleeve 104.

FIG. 41 depicts a compressor/bracket 111/139 installed on the concave curvature of a scoliotic vertebral segment.

FIG. 42 shows disc 100 compression and penetration with time by the compressor 111 to correct or straighten the scoliotic vertebral segment.

FIG. 43 indicates a biodegradable sleeve 218 restricting the elastic struts 107 of the bolt 161 from opening and elastically shortening.

FIG. 44 shows a coil spring 125.

FIG. 45 depicts a coronal view of disc 100 compression by the compressor 111 and coil spring 125 assembly.

FIG. 46 indicates shimming of the compressor 111 into the disc 100 with time, compressed by the coil spring 125.

FIG. 47 shows a spring 124 including of two connecting lift springs 121, which can provide disc compression similar to the coil spring 125.

FIG. 48 indicates a compressor 111 with an elastic fastening means 127 installed at the anterior portion of a kyphosis vertebral segment.

FIG. 49 shows correction of the kyphosis vertebral segment by disc 100 elevation and penetration of the compressor 111.

FIG. 50 depicts a disc compressor 111 on a lengthened bracket 139 designed to fuse the vertebral segment and elevate disc space.

FIG. 51 shows spinal fusion and disc 100 compression with the lengthened compressor/bracket 111/139 fastened with bolts 161 or screws concealed in the indentation 164.

FIG. 52 indicates a coronal view of spinal fusion and disc 100 compression with the lengthened compressor/brackets 111/139 fastened on the vertebral bodies 139.

FIG. 53 indicates a coronal view of normal bulging of annular layers during axial compression.

FIG. 54 shows annular delamination due to inward and outward bulging caused by aging or a dehydrated nucleus pulposus 128.

FIG. 55 depicts seepage of nucleus pulposus 128 through damaged annular layers, possibly from the weakened, delaminated annular layers.

FIG. 56 indicates disc 100 compression by the compressors 111, promoting inward annular bulging to minimize further delamination.

FIG. 57 shows the sinuvertebral nerve 216 ingrowth into the disc 100, causing discogenic pain.

FIG. 58 depicts compression of the sinuvertebral nerves 216 by the compressors 111 to atrophy the nerves 216.

FIG. 59 depicts the insertion of a trocar 103 laterally through the bulging disc 100, with the aid of a guide 185 (optional).

FIG. 60 indicates the insertion of a dilator 230 over the trocar 103.

FIG. 61 shows the withdrawal of the trocar with the dilator 230 remaining in the disc 100.

FIG. 62 depicts the insertion of a bolt 161, compressor 111 and washer 163 assembly into the dilator 230.

FIG. 63 indicates the withdrawal of the dilator to expose the thread 109 of the bolt 161.

FIG. 64 shows the installation of another compressor 111 onto the bolt 161 with washer 163 and nut 162.

FIG. 65 depicts disc 100 compression by tightening the nut 162 on the bolt 161.

FIG. 66 depicts fastening of a compressor/bracket 111/139 with a screw 187 through part of the disc 100 into vertebral body 159, another screw 187 through the bracket 139 into the side of vertebral body 159.

FIG. 67 shows surgically inflicted bleeding sites 224 by a trocar 103 at the end plate 105 for annular adhesion and/or regeneration and a deep puncture for screw entry 217.

FIG. 68 depicts surgically inflicted bleed sites 224 at the end plate 105 by a curved trocar 103.

FIG. 69 shows a screw 187 through a compressor 111 with a trough 223 or indentation to conceal a screw head 226.

FIG. 70 shows the installation of the compressor 111 into the end plate 105 through a protruded disc 100 impinging 184 on a nerve 102.

FIG. 71 shows disc 100 fastening by the compressor 111 to alleviate the impingement of an adjacent nerve 102.

FIG. 72 depicts a coronal view of the compressor 111 fastened through the outer portion of the disc 100 into the end plate 105 with bleeding sites 224 created to promote annular adhesion and regeneration.

FIG. 73 depicts nerve impingement 184 from spondylolisthesis.

FIG. 74 shows surgically inflicted bleeding sites 224 at the end-plate 105 by a trocar 103 to promote adhesion and reattachment between the disc 100 and vertebral body 159.

FIG. 75 depicts a rigid sleeve 220 sliding on an elastically curved trocar 103 with a label 221 on the handle indicating the direction of the curvature.

FIG. 76 shows that the curvature of the elastic trocar 103 is resiliently straightened within the lumen of the sleeve 220.

FIG. 77 demonstrates that the end plate 105 can be reached even when the sleeve 220 is introduced perpendicularly to the disc 100.

FIG. 78 depicts a bulging disc 100 sandwiched between two vertebral bodies 159. The bulges may result in spinal stenosis and/or segmental instability.

FIG. 79 depicts disc 100 compression, stabilization and elevation with two compressors 111 anchored through the end plate 105 into the vertebral body 159.

FIG. 80 shows disc 100 thickening with the fastened compressor 111 to reduce spinal stenosis.

FIG. 81 depicts disc 100 fastening with screws 187 anchoring into vertebral bodies 159, above and below the intervertebral disc 100.

FIG. 82 shows a bolt 161 traversing through the end-plate 105 and the vertebral body 159 to fasten the compressor 111 with a nut 162 supported by a washer 163.

FIG. 83 depicts a compressor 111 with multiple tissue ingrowth openings 160.

FIG. 84 depicts a compressor 111 with outwardly curved tips 130 and tissue ingrowth openings 160 penetrating through the thickness of the compressor 111.

FIG. 85 shows a resilient compressor 111 in an open or predisposed position.

FIG. 86 depicts the resilient compressor 111 being constricted or folded within a delivery capsule 131.

FIG. 87 indicates the insertion of the delivery capsule 131 onto a protruded disc 100.

FIG. 88 shows the advancing screw 187 anchoring in the vertebral body 159 and expelling the compressor 111 from the capsule 131 onto the protruded disc 100.

FIG. 89 indicates disc 100 fastening with the compressor 111 in an expanded or compressed position.

FIG. 90 depicts a stabilizer 134 inserted within the delivering capsule 131 to minimize tilting of the screw head 226 during disc 100 fastening.

FIG. 91 shows a clamp/compressors 198/111 with large tissue ingrowth openings 160.

FIG. 92 shows bone ingrowth from upper and lower vertebral bodies 159 into the tissue ingrowth openings 160 of the clamp/compressors 198/111 leading to spinal fusion 234.

### DETAILED DESCRIPTION OF THE EMBODIMENTS

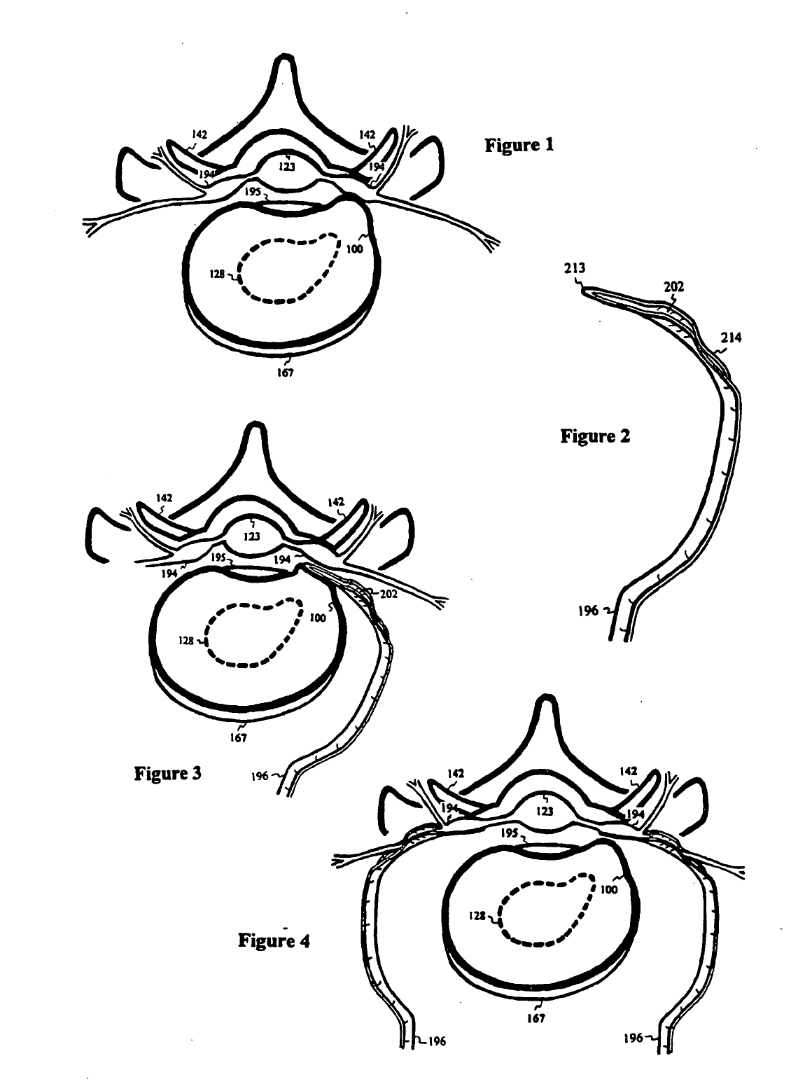

FIG. 1 depicts a common nerve 194 impingement from a protruded disc 100 at or near the narrow channel of the neuroforamen. For protection during disc 100 repair, a nerve shield 196 contains a thin and blunt distal tip 213 for reaching into or near the neuroforamen, a trough 202 to partially surround and protect the nerve 194 and an open channel 214 for the nerve 194 to exit from the trough 202. Through anterior or lateral incision, the nerve shield 196 is introduced by sliding over the bulging annulus of the disc 100, as shown in FIG. 3, to minimize potential damage to the ventral/dorsal ramus nerve root 194. The shield 196 is then gently pressed against the partially surrounded nerve 194. Similarly, another nerve shield 196 is used contralaterally to protect both nerves 194 existing from the neuroforamen, as shown in FIG. 4.

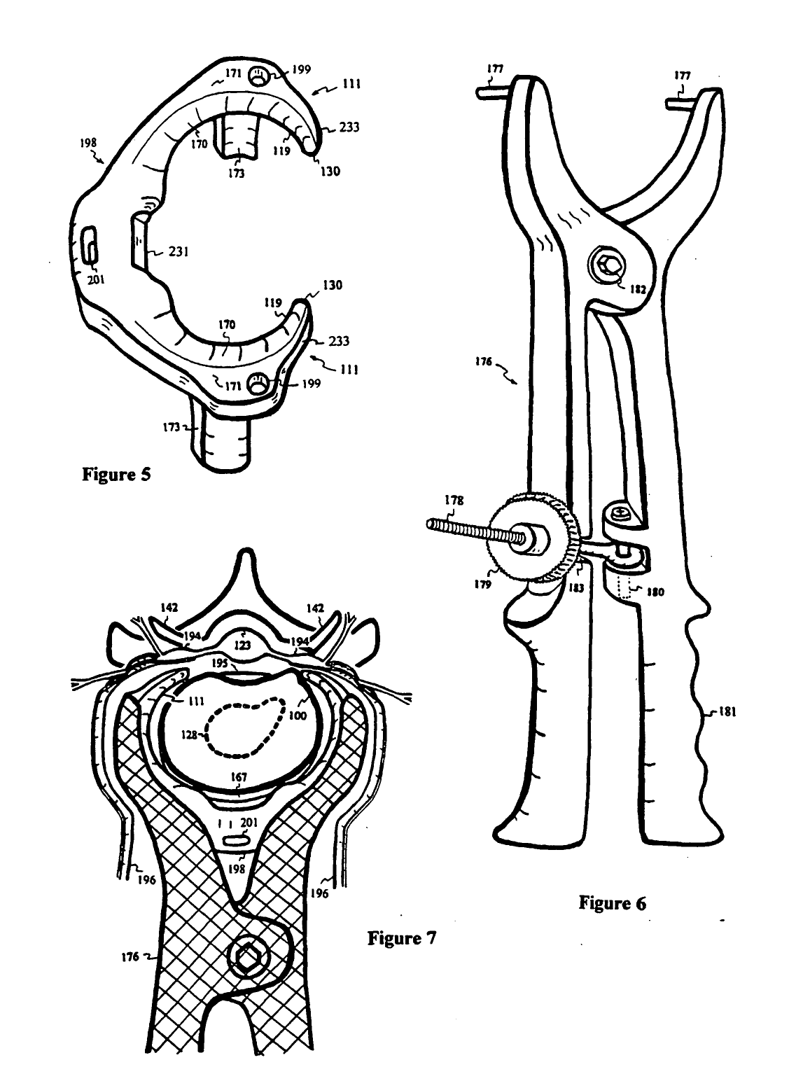

FIG. 5 shows an elastic intervertebral disc clamp 198 with an annular contact surface 119, a sloped surface 170, a plateau surface 171 and stops 173 on the compressors 111 portions. The saddle-shaped compressors 111 are used to bracket the dysfunctional disc 100 bilaterally. The clamp/compressor 198/111 has a support mount 201, an indentation 231 and two widening mounts 199 for engagement with a widening tool, as shown in FIG. 6. The clamp 198 can be made with nickel-titanium, nitinol, or other elastic alloy or polymers. FIG. 6 shows a clamp-widening tool 176 equipped with clamp grabbers 177 for engaging with the widening mount 199 on the compressors 111, a pivotal joint 182, handles 181 and a locking mechanism capable of slowly releasing the compressor 111. The mechanism contains a hinge 180 anchoring a lock screw 178 fastened with a lock wheel 179. The lock screw 178 is sized and configured to fit into a lock slot 183 to lock the handle 181 of the widening tool 176. For quick release of the handle 181, the lock screw 178 can be picked up from the slot 183. For slow release, the lock wheel 179 can be rotated to slowly open the handle 181, thus slowly closing the disc clamp 198.

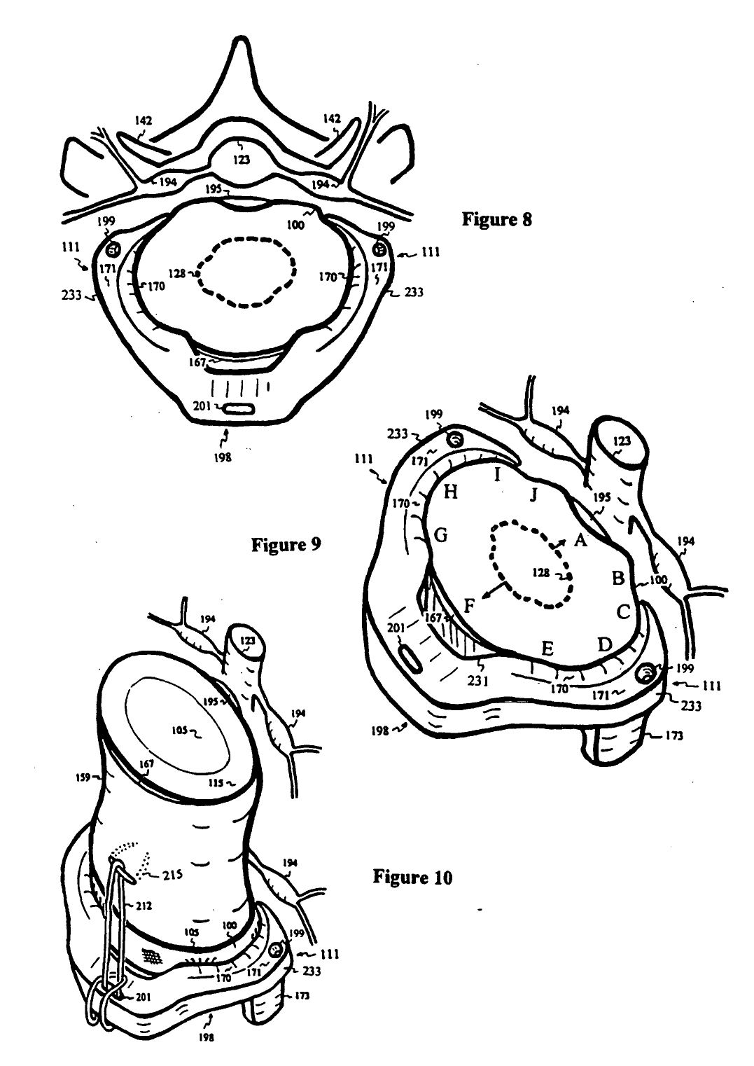

FIG. 7 depicts widening and placement of the disc clamp 198 by the widening tool 176. The clamp 198 fits around the intervertebral disc 100, while nerves 194 are protected by nerve shields 196. The distal tips of the compressors 111 are thin and tapered to prevent impingement of the nerve 194. The clamp 198 is then slowly released by dialing the lock wheel 179, as shown in FIG. 6. FIG. 8 shows the disc 100 being clamped by the disc clamp 198 as the compressors 111 press the bulging annulus inwardly to alleviate nerve 194 impingement. The size of the clamp/compressor 198/111 is enlarged disproportionately to the disc 100, for clarification. FIG. 9 indicates the locations of compression from the disc clamp 198. The preferred compressions are at areas C and I, common protruding locations of the disc 100, with areas E and G as supporting locations. From a disc 100 fastening cadaveric study, nearly the entire disc 100 was distracted, elevated and slightly lengthened from compression by the compressors 111. The portion of annulus remote to the compressors 111 was also distracted, pulling inward. The previously protruded areas B and J in FIG. 9 would similarly be distracted as well. Annulus distraction is wide spread and far reaching, way beyond the area of direct compression. The benefit of the far-reaching capability of the compressors 111 is most significant in repairing annular impingements commonly occurring around the narrowed neuroforamen. The compressors 111 can be fastened a distance away from the impinging neuroforamen, yet the distraction of the annulus can draw in the distant bulge, alleviating the impingement. Alternatively, decompressing the nerve impingement within the neuroforamenal region (the hidden zone) surrounded by the disc 100, vertebral body 159, pedicle and facet joint 129 is very invasive using current surgical procedures, and it may result in increased scarring and a permanently weakened spine.

As the disc 100 is compressed by the body weight, area F located at the indentation 231 and area A are allowed to naturally and resiliently bulge as indicated by arrows in FIG. 9, since they are least restricted by the clamp 198. The thinning or tapering of the distal tips of the compressors 111 are essential to avoid nerve 194 impingement, as shown in FIGS. 8 and 9. To minimize possible damage to the disc 100, the annular contact surfaces 119 of the compressors 111 are generally cylindrical or blunt, thickening into the sloped surface 170, as shown in FIG. 5, with an optional plateau surface 171.

To prevent migration of the clamp 198, especially during initial installation, an elastic strap 212 is threaded through the support mount 201 and secured by a staple 215 anchored in the vertebral body 159, as shown in FIG. 10. More than one strap 212 and staple 215 can be used. The strap 212 can be a biodegradable suture or material to initially secure the clamp 198 until the sloped surfaces 170 of the compressors 111 penetrate the annulus and adequately secure the clamp/compressors 198/111.

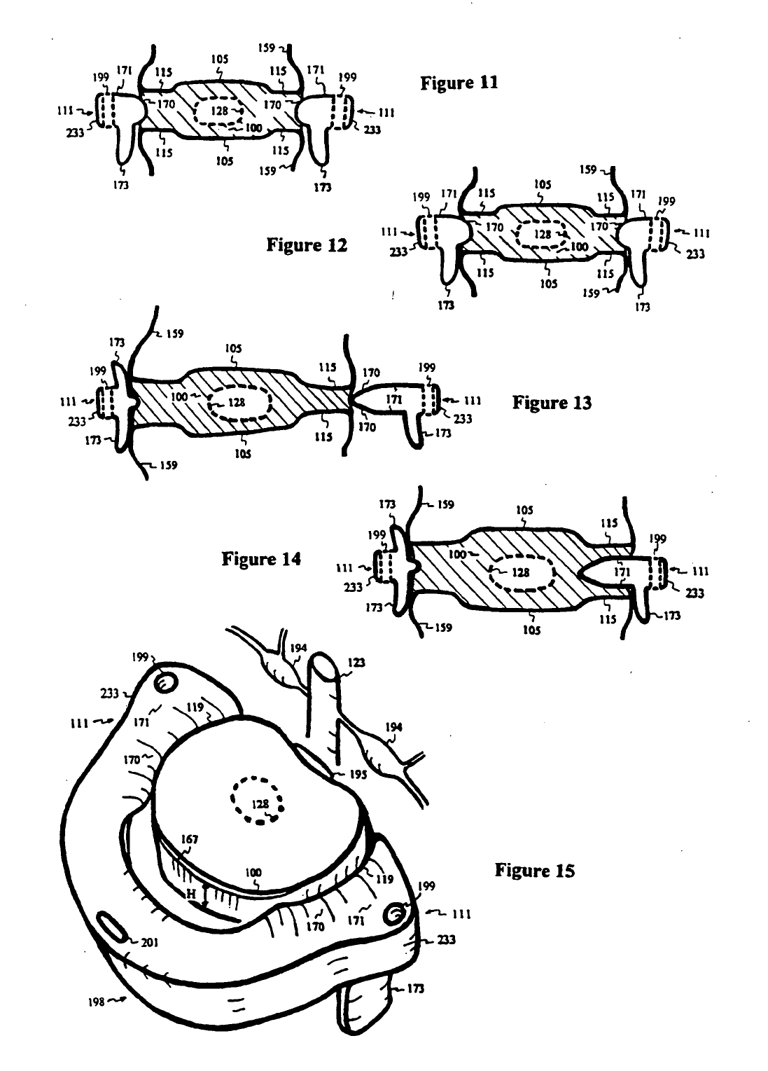

FIG. 11 depicts a coronal view of initial clamping of the disc 100 with the sloped surface 170 resting on the disc 100. With time, the sloped surface 170 of the compressor 111 slowly penetrates into the disc 100 until the stops 173 gently rest on the lateral side of the vertebral body 159 below the disc 100, as shown in FIG. 12. The stop 173 is a protrusion, a small wall or a leg from the under side of the compressor 111. The clamp/compressors 198/111 is designed to compress the protruded annulus, alleviating the nerve impingement. The clamp/compressors 198/111 also restricts, support and stabilize the bulging annulus to alleviate pain from segmental instability.

Current surgical treatment for scoliosis is invasive, most frequently done on young female patients to correct the deformity. Instrumentation failure or breakage of pedicle screws is likely after decades of wear and tear, mandating a second surgery. FIG. 13 depicts a coronal view of a scoliotic vertebral segment initially clamped and compressed by the unsymmetrical compressors 111 of a disc clamp 198 (not shown). The concave side of the curved vertebral segment is fitted with a thick compressor 111 comprising a wide plateau surface 171, while the convex side of the vertebral segment is fitted with a thin compressor 111 containing a narrow or absent plateau surface 171. FIG. 14 shows correction or straightening of the scoliotic vertebral segment with time, by selectively wedging, shimming and elevating the concave side of the curved vertebral segment and by inserting the plateau surface 171 of the compressor 111 between the dense epiphyses 115. To straighten the entire spine, multiple selective disc 100 elevations are required, much as multiple pedicle screws and instrumentation are used in current procedures. Scoliosis is corrected through selective shimming by the compressor 111 to alter the lateral curvature of the spine. Nickel-titanium compressors 111 are expected to be durable between the epiphyses 115; and the clamp 198 is under minimal strain after settlement in the disc 100. Thus the clamp/compressors 198/111 are expected to be long lasting, perhaps even permanent without revisional surgery.

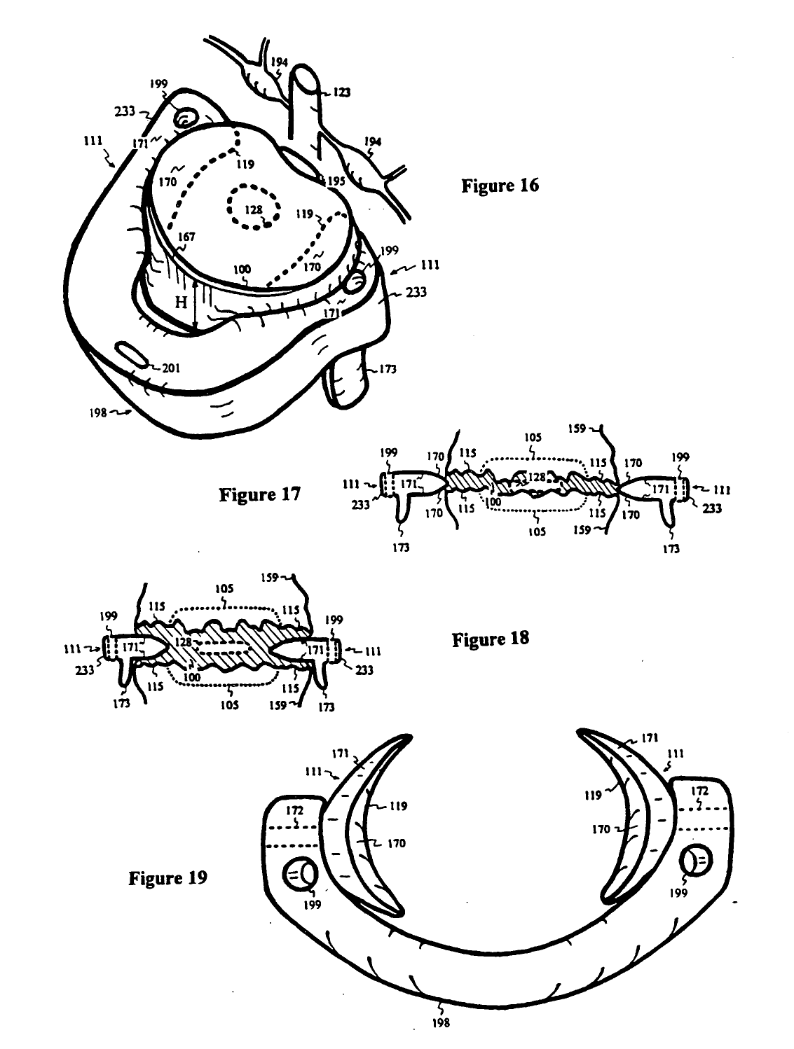

Spinal stenosis is a progressive disorder. FIG. 15 depicts a flattened disc 100 with a dehydrated nucleus pulposus 128. The initial disc height, H, is indicated at the anterior portion of the disc 100. A clamp 100 with two symmetrical compressors 111 with wide plateau surfaces 171 is clamped around the flattened disc 100. The size of the clamp/compressors 198/111 is enlarged disproportionately to the disc 100, for clarification. Gentle compression and wedging action of the clamp/compressors 198/111 allow time for the annulus to grow and thicken. The surrounding ligaments, including the posterior 195 and anterior 167 longitudinal ligaments and facet joint ligaments, also require time to lengthen. As the sloped surface 170 wedges into the disc 100, the plateau surfaces 171 establish stable positions between epiphyses 115 to thicken the disc 100 and provide elevated disc height, H, as shown in FIG. 16. With elevated intervertebral disc space, nerve impingement caused by spinal stenosis is minimized or alleviated. Disc 100 penetration by the compressors 111 halts when the stops 173 reach the lateral surfaces of the vertebral body 159, in this case below the disc 100. FIG. 17 depicts a coronal view of a clamp 198 (not shown) and compressors 111 initially clamped around a disc 100 sandwiched by bone spurs, common among patients with spinal stenosis. With time, FIG. 18 shows wedging and penetration of the sloped surfaces 170 followed by the plateau surfaces 171 into the disc 100 between the epiphyses 115 of the vertebral bodies 159. Thus, disc 100 height increases to alleviate nerve impingement common among spinal stenosis patients. Penetration of the compressors 111 halts when the stops 173 rest upon the vertebral body 159 below the disc 100. The plateau surface 171 maintains disc height without the need of further compression. In contrast to current surgical techniques, which cut or bur away anatomical structure to make room for the progressively narrowing disc space, the clamp/compressors 198/111 restore or increase the disc 100 height to minimize or alleviate nerve impingement.

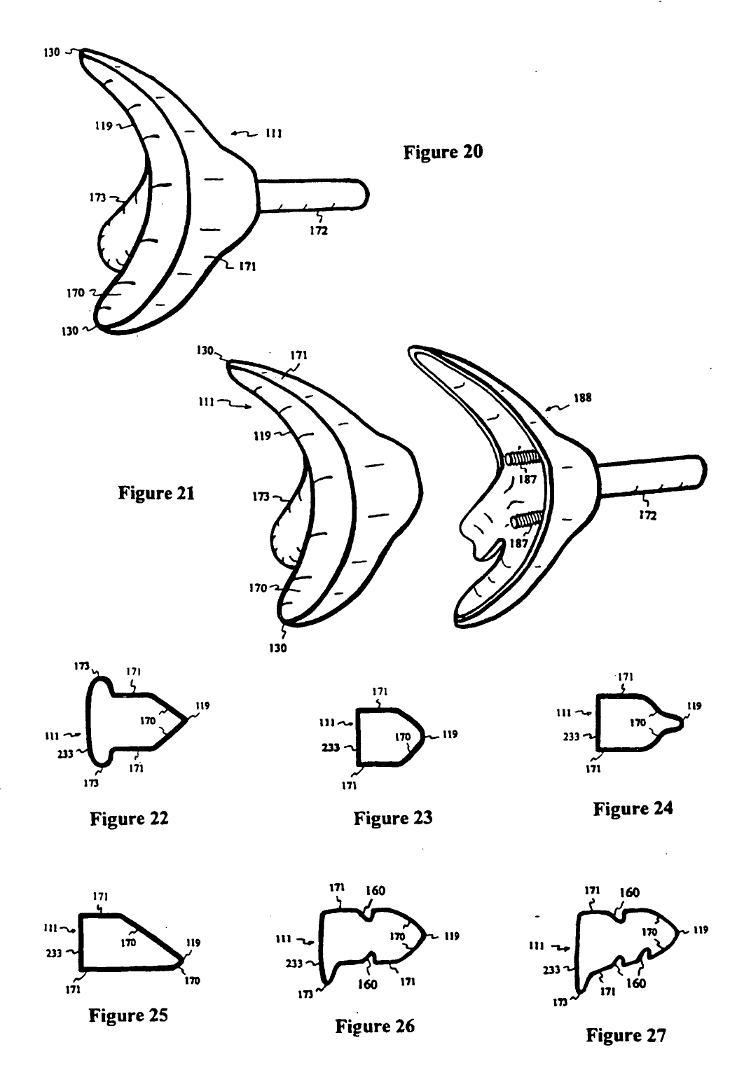

The clamp 198 and the compressors 111 can be made separately as modular components assembled into a device as shown in FIG. 19. The vertical cross-section of the clamp 198 can be semi-circular, elliptical, circular or another shape with blunt surfaces to prevent abrasion to the disc 100, abdominal contents or blood vessels. The saddle-shaped compressor 111 contains a pivotal peg 172 for inserting into the clamp 198, a smooth and blunt annular contact surface 119, a sloped surface 170, a plateau surface 171 and a stop 173, as shown in FIG. 20. The concave curvature of the annular contact surface 119 of the compressor 111 is designed to conform and fit partially around the disc 100. Since most discs 100 are not circular, the concave or crescent curvature of the annular contact surface 119 is likely to be complex or to contain multiple radiuses in order to conform to the surface of a disc 100. One of the tips 130 of the compressor 111 is particularly thin and tapered, designed to minimize nerve impingement especially near the neuroforamen. The compressor 111 can also be made with modular components, as shown in FIG. 21. The annular contacting part of the compressor 111 can be made with biocompatible polymer, such as polyurethane, polypropylene, polyethylene, PEEK, Delrin, polysulfone, polytetrafluoroethylene, polycarbonate, ultra high molecular weight polyethylene or other low friction polymer. The casing 188 with pivotal peg 172, as shown in FIG. 21, can be made with stainless steel, titanium, nickel-titanium or metal, or even a polymer. The components can be assembled with screws 187 also shown in FIG. 21.

The thickness, curvature, surfaces 119, 170, 171 and/or stops 173 of the compressor 111 can vary to accommodate proper disc 100 compression. FIG. 22 depicts a vertical cross-sectional view of a compressor 111 containing two stops 173 to improve stability. FIG. 23 shows a compressor 111 with no stop 173 and a round annular contact surface 119 for gentle compression. FIG. 24 indicates a compressor 111 with multiple sloped surfaces 170 to gain rapid annular penetration and provide initial stabilization of the clamp 198. FIG. 25 shows an unsymmetrical slope 170 for shimming into a disc 100 to correct or straighten some kyphosis, scoliosis, lordosis or other spinal deformity. FIGS. 26 shows tissue ingrowth openings 160, indentations or troughs to promote annular ingrowth and stabilization of the compressor 111. The plateau surfaces 171 with tissue ingrowth openings 160 can also be non-parallel to each other, as shown in FIG. 27, to correct and stabilize some spinal deformities.

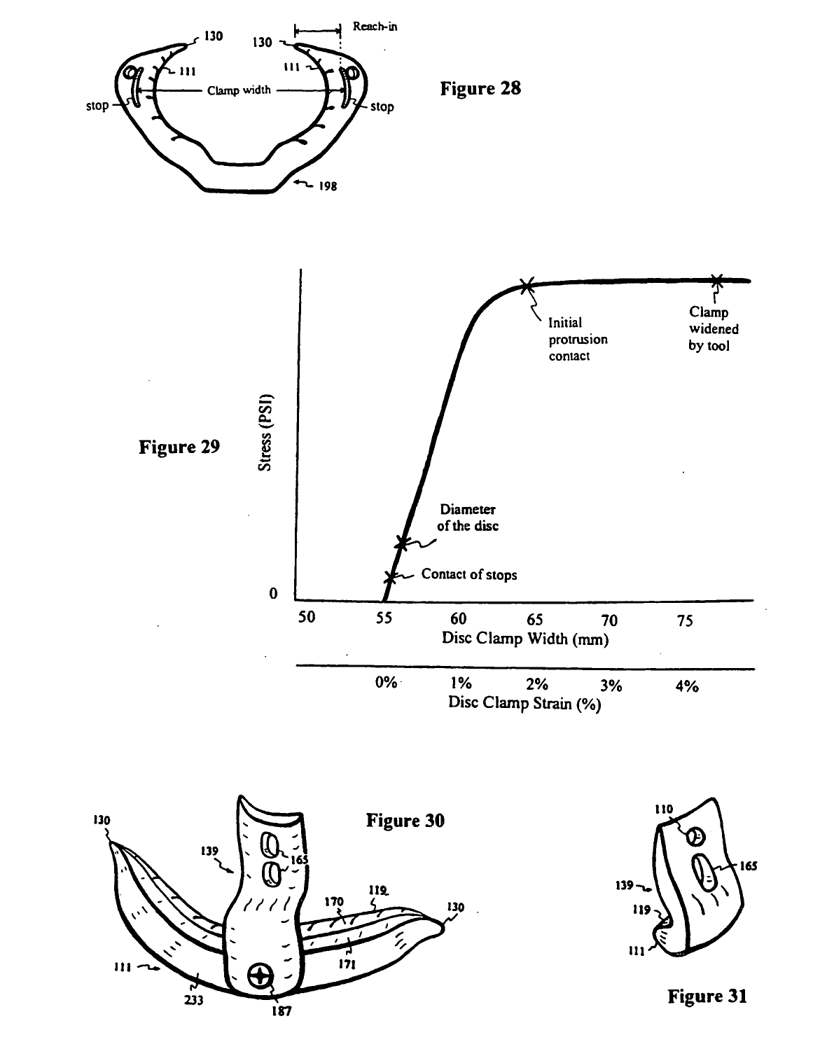

For compressive strength, biocompatibility and durability, nickel-titanium perhaps is the most suitable material for fabricating the clamp 198. The clamp width and reach-in portions are defined in FIG. 28. The reach-in portions of the clamp 198 are essential for securing the initial fastening and clamping of the disc 100. The distal tips 130 are tapered to prevent nerve impingement by the reach-in portions of the clamp 198. FIG. 29 is a typical strain vs. stress profile of nickel-titanium alloy, a super elastic alloy suitable for fabricating into a disc clamp 198. Various compressive stages of a nickel-titanium clamp 198 are also indicated in FIG. 29. The compressive force is greatest initially when it presses in the annular protrusion. As the protrusion is compressed, it relieves the strain of the clamp 198; the compressive force of the clamp 198 rapidly weakens. When the stops 173 reach the vertebral body 159, the compressive force is insignificant, minimizing erosion on bone and annulus. Since the stress on the clamp 198 is minimal after protrusion compression, continual erosion of the disc 100 may not occur even in the absence of the stops 173 on the compressors 111.

The clamp/compressors 198/111 can also be installed through a lateral incision. A widening tool is modified to hold the clamp/compressors 198/111 laterally. The modified tool is also used as an extension to install the device 198/111 in the patient. Lateral insertion and device 198/111 maneuvering can minimize possible damages from excessive tissue retraction, especially for intervertebral discs 100 surrounded by blood vessels, muscles and nerves. For example, the L3-4 disc 100 is sandwiched bilaterally by the Psoas major muscles containing lumbosacral nerve roots, sensitive to excessive retraction. Aorta and inferior vena cava are anterior to the disc 100. To compress the L3-4 disc 100, the open side of the widened C-like clamp/compressors 198/111 is oriented vertically either superiorly or inferiorly to the patient, to make the insertion as thin as possible. Through a lateral incision, the widened and vertically oriented C-like clamp/compressors 198/111 is inserted between the L3-4 disc 100 and the blood vessels (aorta and inferior vena cava) anterior to the disc 100. The clamp/compressors 198/111 is then slowly rotated to orient the open side posteriorly, placing both compressors 111 laterally around the L3-4 100. The clamp/compressors 198/111 is then slowly released to compress the disc 100, followed by retrieval of the widening tool.

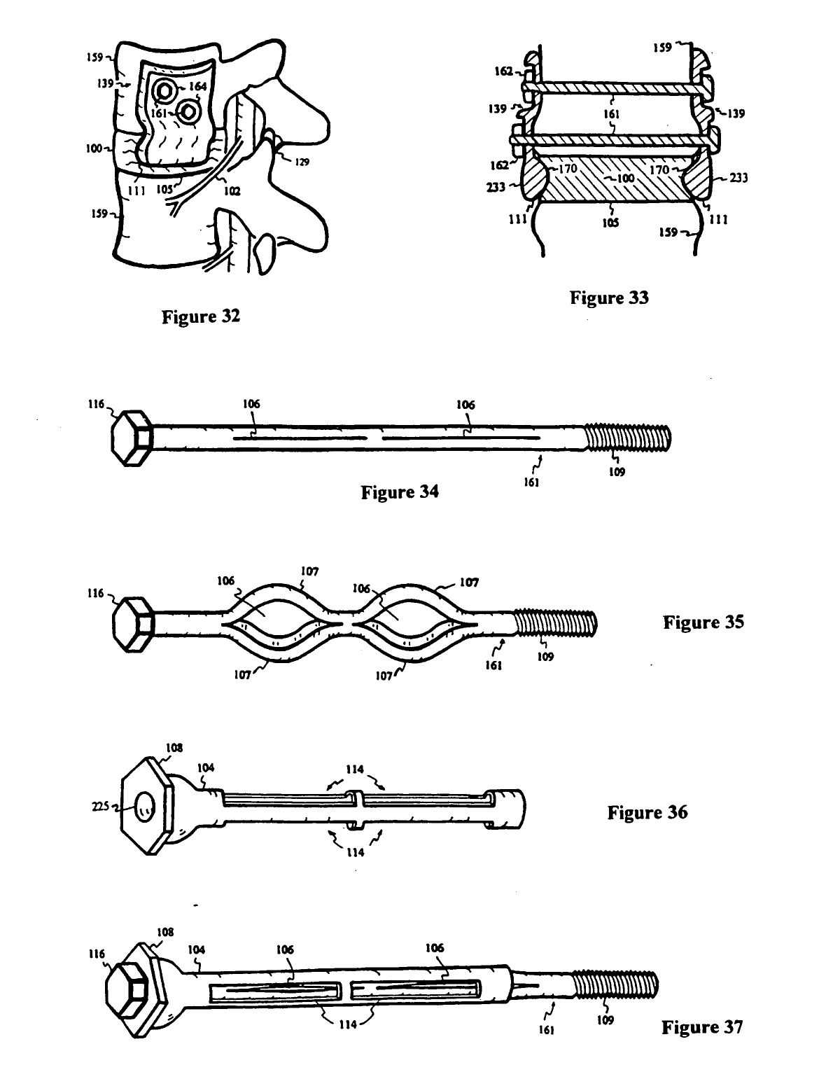

The compressor 111 can also be fastened to a bracket 139 by a screw 187, as shown in FIG. 30. The bracket 139 is equipped with slits 165 for bolts or screws to fasten into the vertebral body 159, thus compressing the protruded disc 100 with the compressor 111. The compressor 111 can also be made with the bracket 139 in one-piece as shown in FIG. 31. FIG. 32 depicts compression of the protruded disc 100 by the compressor/bracket 111/139 fastened by bolts 161 or screws into the vertebral body 159 with the heads of the bolts concealed in the indentation 164 of the bracket 139. FIG. 33 shows a coronal view of bilateral disc 100 compression fastened with compressor/bracket 111/139 and bolts 161 through the vertebral body 159. In essence, the brackets 139 serve similar function as the stops 173 with attachment holes 165, 110.

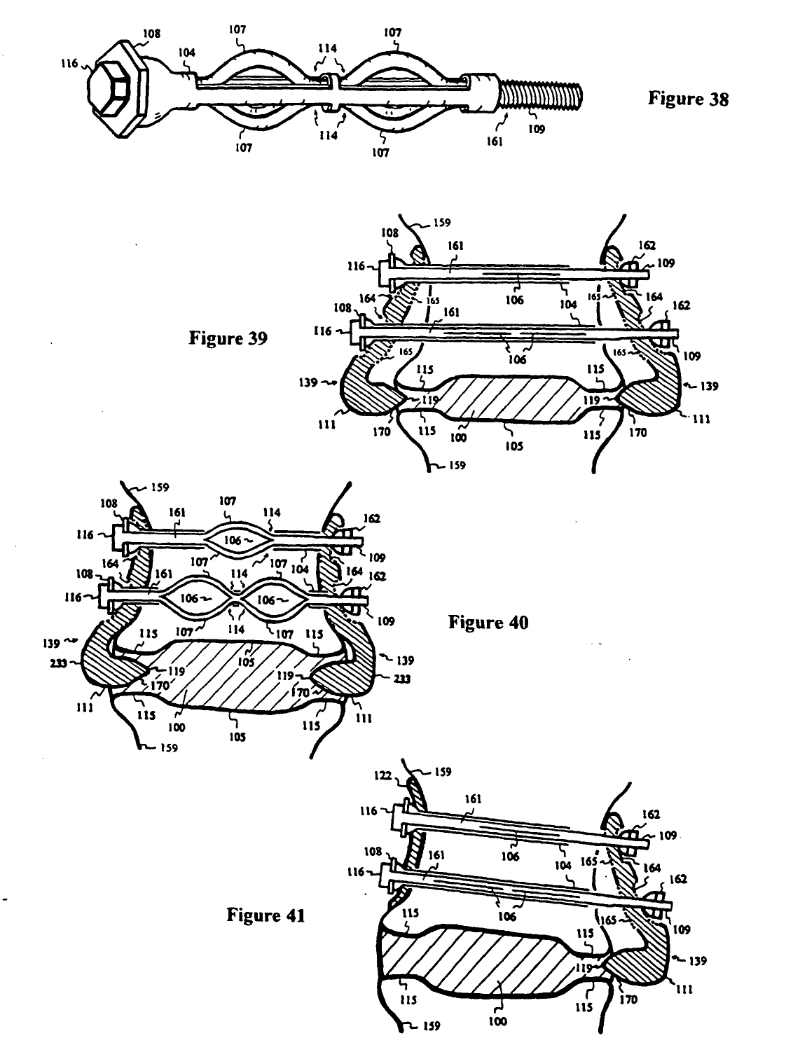

FIG. 34 depicts a bolt 161 with two longitudinal slits 106 cut along the length of the bolt 161. The bolt 161 is made with elastic metal, such as nickel-titanium. The slits 106 can be cut with laser, water jet, wire or sinker EDM (electron discharging machine). FIG. 35 depicts the slits 106 after being shimmed open and shaped to form four elastic and compressible struts 107. For nickel-titanium bolts 161, the struts 107 are shaped by inserting shims or fixtures, heating the shimmed bolts 161 to about 500° C. for 5-10 minutes, then quickly quenching the heat-treated bolt 161 in cold water before removing the fixtures. It is also possible to mold or cast a bolt 161 with elastic and compressible struts 107 already in open positions, as shown in FIG. 35. Elastic polymers can also be used to mold into an elastic bolt 161 with compressible struts 107. With the struts 107 open, the length of the bolt 161 is elastically or resiliently shortened. FIG. 36 shows a sleeve 104 with lumen 225 and four windows 114 sized and configured for the protrusion of the elastic struts 107 of the bolt 161. FIG. 37 indicates the insertion of the bolt 161 with the elastic struts 107 being resiliently compressed and fitted within the sleeve 104. The struts 107 and the windows 114 are in an out-of-phase position, where the windows 114 and direction of struts 107 deployments do not overlap. The length of the bolt 161 in out-of-phase position within the sleeve 104 is longer than the length of the bolt 161 with open struts 107, as shown in FIG. 35. FIG. 38 depicts turning of the bolt 161 relative to the sleeve 104 or turning of the sleeve 104 relative to the bolt 161, from the out-of-phase position to an in-phase position, where the windows 114 align with the directions of struts 107 for deployment. As a result, the elastic struts 107 protrude out of the windows 114 and the overall length of the bolt 161 is elastically or resiliently shortened.

FIG. 39 shows a coronal view of a vertebral motion segment with decreased disc height or symptoms of spinal stenosis. Two disc-compressor/brackets 111/139 are laterally anchored with two elastic bolts 161 containing slits 106 within two sleeves 104 in out-of-phase positions. The round sleeve head 108 and round nut 162 are designed to allow pivotal movement of the compressor/brackets 111/139 during disc 100 compression. The deployment of the struts 107 is activated or initiated by rotating the sleeves 104 from out-of-phase to in-phase positions, allowing the struts 107 to protrude out of the windows 114 of the sleeves 104 and to provide elastic or resilient inward pulling tension on both compressors/brackets 111/139. Similar to the clamp/compressor 198/111, the elastic disc 100 compression allows time for the surrounding ligaments to slowly extend and the annulus of the disc 100 to gradually thicken. As a result, tissue damage is minimized and disc 100 height is elevated to alleviate spinal stenosis, as indicated in FIG. 40. For ease of illustration, FIG. 40 shows that the plane of the deployed struts 107 is perpendicular to the end plate 105, but ideally the plane of the deployed struts 107 should be parallel to the end plate 105 to maximize the spread of the struts 107 without interfering with the end-plate 105. Therefore, a marking on the bolt head 116 visible to the surgeon can be helpful to identify the plane of struts 107 deployment.

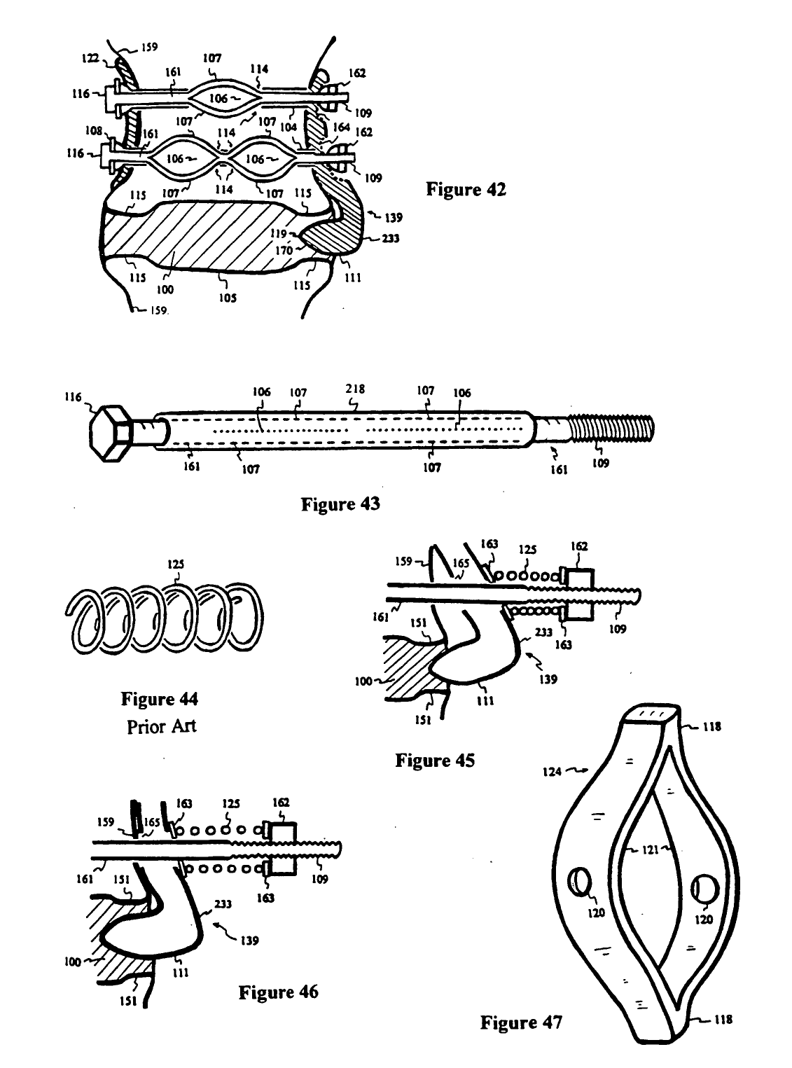

FIG. 41 depicts a mono-lateral disc 100 compression into the concave side of the curved scoliotic vertebral segment. FIG. 42 shows activation of elastic fastening by setting the bolt 161 and sleeve 104 to the in-phase position, slowly wedging the compressor 111 into the concave side of the curved spine to correct or straighten the scoliotic vertebral segment. To correct the entire scoliotic spine, multiple shillings can be done in multiple scoliotic segments. The degree of individual shimming can be individually selected or fitted with different thicknesses and shapes of the compressor 111. The plateau surfaces 171 of the compressor 111 can be non-parallel, as shown in FIG. 27, to optimize the fit and correction. The plateau surfaces 171 can also be indented with a tissue ingrowth opening 160, also indicated in FIG. 27, to promote annular ingrowth and minimize outward slippage of compressor 111.

FIG. 43 indicates a degradable sleeve 218 holding or restricting the elastic struts 107 of the bolt 161 from opening. The rate of strut 107 opening is determined by the rate of degradation of the degradable sleeve 218. The major benefit to the degradable sleeve 218 is the elimination of the step of turning from the out-of-phase to the in-phase position. Furthermore, gradual opening of the struts 107 may be preferred with a slowly eroding degradable polymer to gently and gradually compress and shim into the disc 100. The degradable sleeve 218 can be made with polylactide, polyglycolide, poly(lactide-co-glycolide), polycaprolactone, polydioxanone, polyanhydride, trimethylene carbonate, poly-beta-hydroxybutyrate, polyhydroxyvalerate, poly-gama-ethyl-glutamate, poly(DTH iminocarbonate), poly(bisphenol A iminocarbonate), poly-ortho-ester, polycyanoacrylate and polyphosphazene. There are natural biodegradable materials, including collagen, gelatin, cellulose, chitin and dextran. Many of these biodegradable materials are not biocompatible in bone or in disc 100. However, the elastic bolt 161 and the degradable sleeve 218 combination can be used in other industries to provide elastic tensile fastening. The degradation can be initiated by water. For implant use, polylactide, polyglycolide or poly(lactide-co-glycolide) is most promising for making the degradable sleeve 218.

It is possible to have both elastic bolt 161 and sleeve 218 biodegradable for bone joining or tissue fastening. Degradation time for DL-polylactide is 12-16 months; 50/50 lactide and glycolide co-polymer is 1-2 months. The bolt 161 with open struts 107 can be made by injection molding with DL-polylactide (modulus 1.9 Gpa) and the sleeve 218 with 50/50 lactide and glycolide. Initiated by the degradation of the sleeve 218 within two months, the resilient strength of the bolt 161 begins. After 16 months, hopefully the wound has healed and the bolt 161 and nut 162 will also degrade.

Similar to the elastic bolt 161, a coil spring 125 as shown in FIG. 44 can also provide compression onto the compressor/bracket 111/139. FIG. 45 depicts a coronal view of disc 100 compression by a bolt 161, compressor/bracket 111/139, washer 163, compressed coil spring 125, another washer 163 and nut 162. FIG. 46 shows disc 100 compression and compressor 111 shimming activated by the coil spring 125. Other type of springs can also be used. FIG. 47 shows two connecting lift springs 121 curving or arching outwardly. The springs 121 are connected at both ends 118, and a screw hole 120 lies near the center of both springs 121. The lift springs 121 can be used as the coil spring 125 in FIGS. 45 and 46 to elastically compress the intervertebral disc 100.

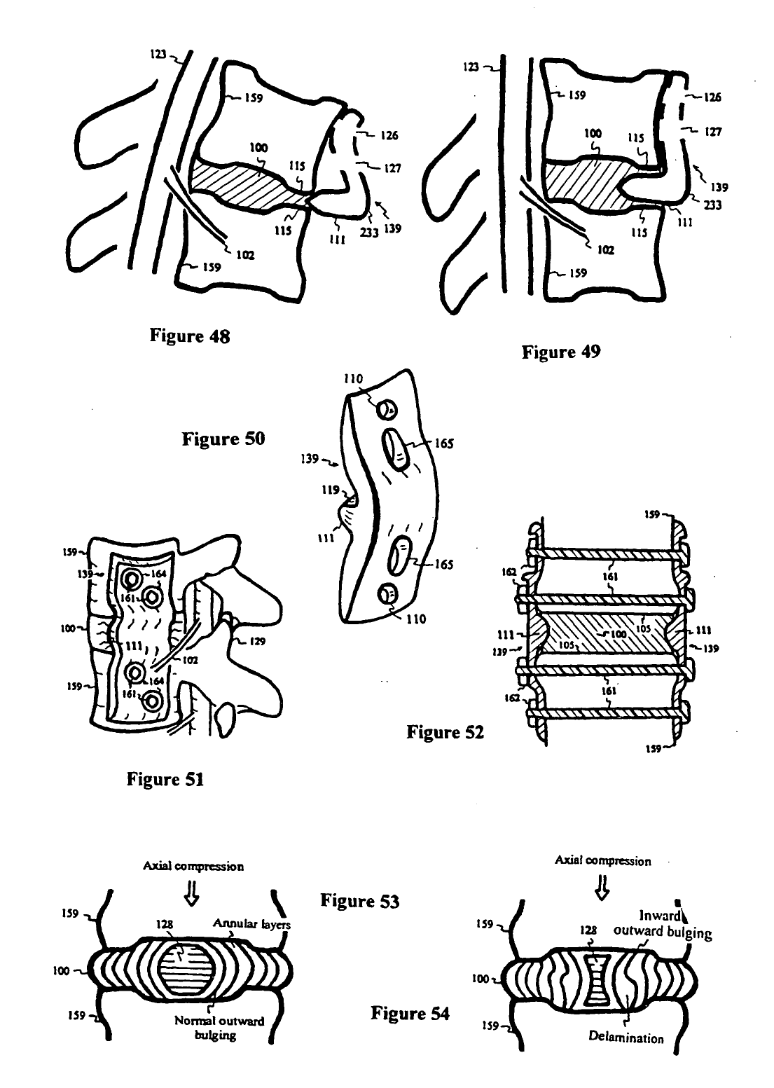

FIG. 48 indicates a compressor/bracket 111/139 installed anterior to a kyphotic vertebral segment. The bracket 139 is anchored by a pivoting means 126 and an elastic fastening means 127 onto the vertebral body 159. With time, the compressor 111 shims into the disc 100 to correct and straighten the kyphotic bend as shown in FIG. 49. The bracket 139 can also be made with elastic or resilient material installed under strain to compress into the disc 100.

The compressor/bracket 111/139 can also be lengthened to serve dual functions: disc 100 compression and spinal fusion, as shown in FIG. 50. Differing from the currently existing fusion plate, the extended compressor/bracket 111/139 compresses and thickens the disc 100 to increase disc space and possibly alleviate nerve impingement. The extended bracket 139 contains a compressor 111 near the mid-portion and screw/bolt holes 110 or slits 165 above and below the compressor 111. FIG. 51 depicts spinal fusion and disc compression with the extended compressor/bracket 111/139. A coronal view of spinal fusion and disc compression with two compressors/brackets 111/139 fastened on the vertebral bodies 159 is shown in FIG. 52. For the best results, the bolts 161 or screws are fitted in the slits 165 and evenly fastened to compress the disc 100 and distract the vertebral bodies 159. Then holes are then created in the vertebral bodies to fit bolts 161 or screws through the bracket holes 110 and to further secure the bracket 139. Disc 100 compression with spinal fusion is expected to provide disc height elevation, which may be particularly suitable for severe segmental instability or spinal stenosis. Using current technique, disc heights commonly decrease after intervertebral body fusion (Watkins R., et. al., Comparison of Disc Space Heights after Anterior Lumbar Interbody Fusion, Spine 14(8):876-878, 1989).

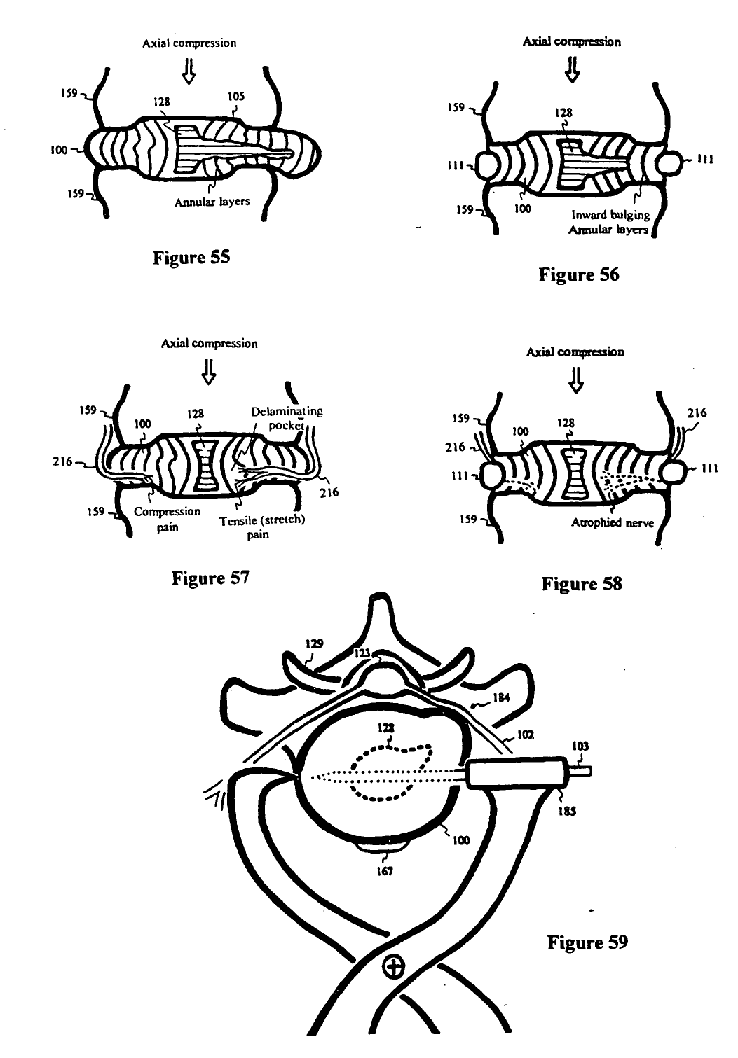

FIG. 53 depicts a mid-coronal view of a vertebral segment with normal outward bulging of the annular layers during axial compression. As the nucleus pulposus 128 ages, dries out or degenerates, the annular layers exhibit both inward and outward bulging during similar axial compressions (Seroussi R. E. et. al., Internal Deformations of Intact and Denucleated Human Lumbar Discs Subjected to Compression, Flexion, and Extension Loads, Journal of Orthopaedic Research, 7:122-131, 1989; Meakin J. R., Replacing the nucleus pulposus of the intervertebral disc, Clinical Biomechanics 16:560-565, 2001). It is speculated that the inward-outward bulging causes delamination in the inner core of the annular layers, as shown in FIG. 54. The delaminated annular layer is thin, unsupported and vulnerable to tearing. Usually, the delamination begins at the layers near the aging nucleus pulposus 128 and leads to seepage of nucleus pulposus 128 and disc 100 protrusion, as shown in FIG. 55, (Goel V. K. et. al., Interlaminar Shear Stresses and Laminae Separation in a Disc, Spine, 20(6): 689-98, 1995). The compressors 111 provide inward compression to the disc 100, flatten the protrusion and promote inward bulging to minimize the progression of annular delamination and to halt the deterioration of the defective disc 100, as indicated in FIG. 56. Disc 100 compression by the compressor 111 may also collapse and seal the seeping channels of nucleus pulposus 128 in a herniated disc 100 to minimize chemical irritation to nerves 102.

Chronic low back pain is generally thought to be caused by nerve 102 impingement. However, MRI often fails to show impingement of neural structures, even in the presence of sciatica. Furthermore, saline injection, discography and compression of the longitudinal spinal ligaments can reproduce back pain and sciatica. These observations have led to re-examination of the pathways and distribution of nociceptive (pain sensing) nerve endings in healthy and diseased spines. In the healthy disc 100, only the outer third of the annulus is innervated. But among patients with chronic low back pain, nerves extend into the inner third of the annulus, some even into the nucleus pulposus 128 (Freemont A. J. et. al., Nerve ingrowth into diseased intervertebral disc in chronic back pain, The Lancet, Vol. 350, July 19:178-181, 1997). Nerve ingrowth in connective tissue is normally a sign of repair in progress. However, similar to the articular cartilage in joints, the healing progress of annulus is very slow and poor. FIG. 57 depicts the ingrowth of sinuvertebral nerves 216 conducting the sensation of tensile or stretching pain from the delaminated pockets within the degenerating disc 100. Sinuvertebral nerves 216 normally grow from the surface into the annulus only when the disc 100 begins to degenerate. FIG. 58 depicts compression of the sinuvertebral nerves 216 leading into the degenerative disc 100 by the compressors 111. With prolonged and intense compression from the compressors 111, the sinuvertebral nerves 216 are expected to cease transmitting signals of pain from the degenerative disc 100 and atrophy within days, thus alleviating pain without discectomy.

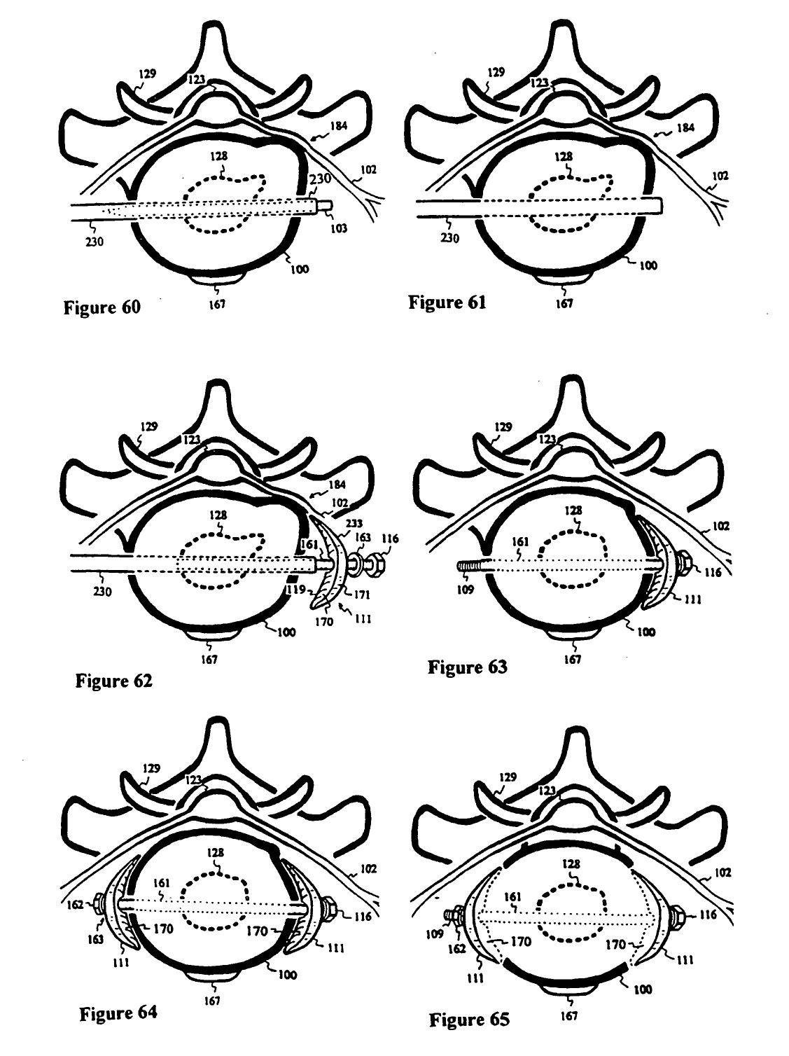

The compressors 111 can also be installed through a protruded disc 100. With the aid of a trocar guide 185, FIG. 59 depicts the insertion of a trocar 103 laterally through the protruded disc 100 impinging 184 upon a nerve 102. Insertion of the trocar 103 and compressors 111 can be done endoscopically through a lateral incision as well as through the anterior approach shown in FIG. 59. FIG. 60 indicates the insertion of a dilator 230 over the trocar 103. Then the trocar 103 is withdrawn while the dilator 230 remains in the disc 100, as shown in FIG. 61. FIG. 62 depicts the insertion of a bolt 161, an arcuate compressor 111 and washer 163 assembly into the dilator 230. FIG. 63 indicates the withdrawal of the dilator 230 to exposure the thread 109 of the bolt 161. FIG. 64 shows the installation of another compressor 111 onto the bolt 161 with washer 163 and nut 162. FIG. 65 depicts tightening of the bolt 161, nut 162, compressors 111 and washer 163 assembly to fasten the bulging disc 100 with the sloped surface 170 embedding into the disc 100. For elastic compression, the resilient bolt 161 with elastic struts 107 can be used with the sleeve 104, as shown in FIG. 37, or with the biodegradable sleeve 218 in FIG. 43.

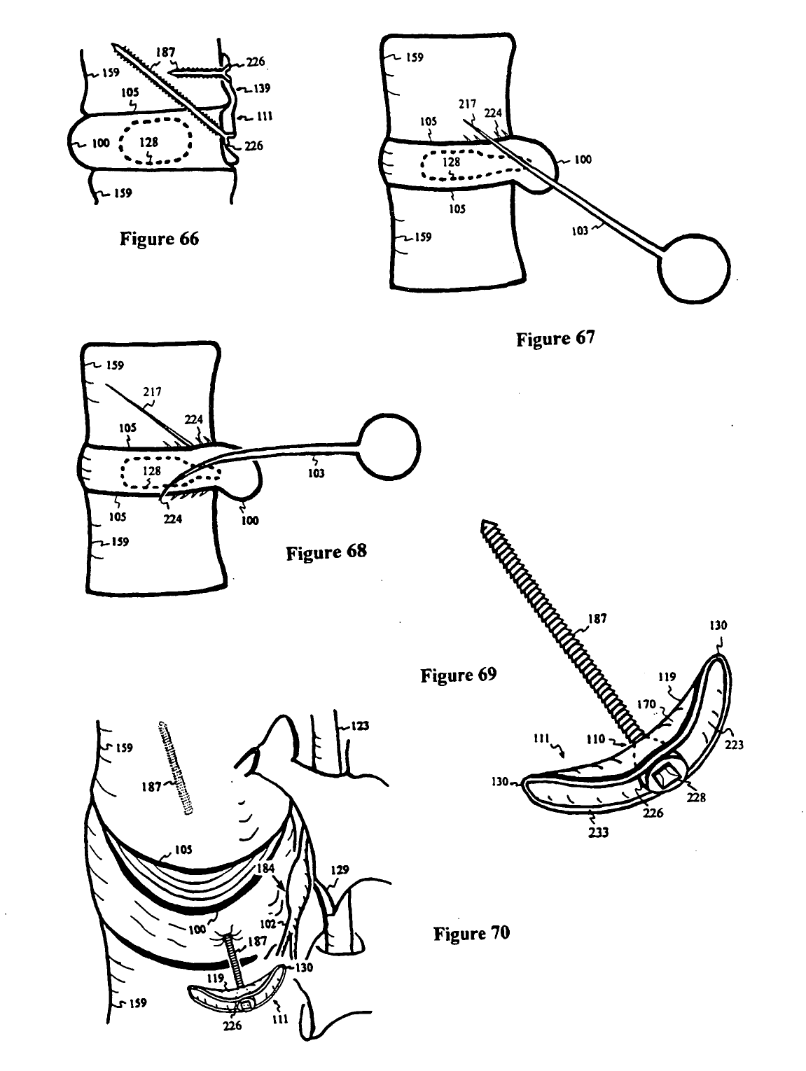

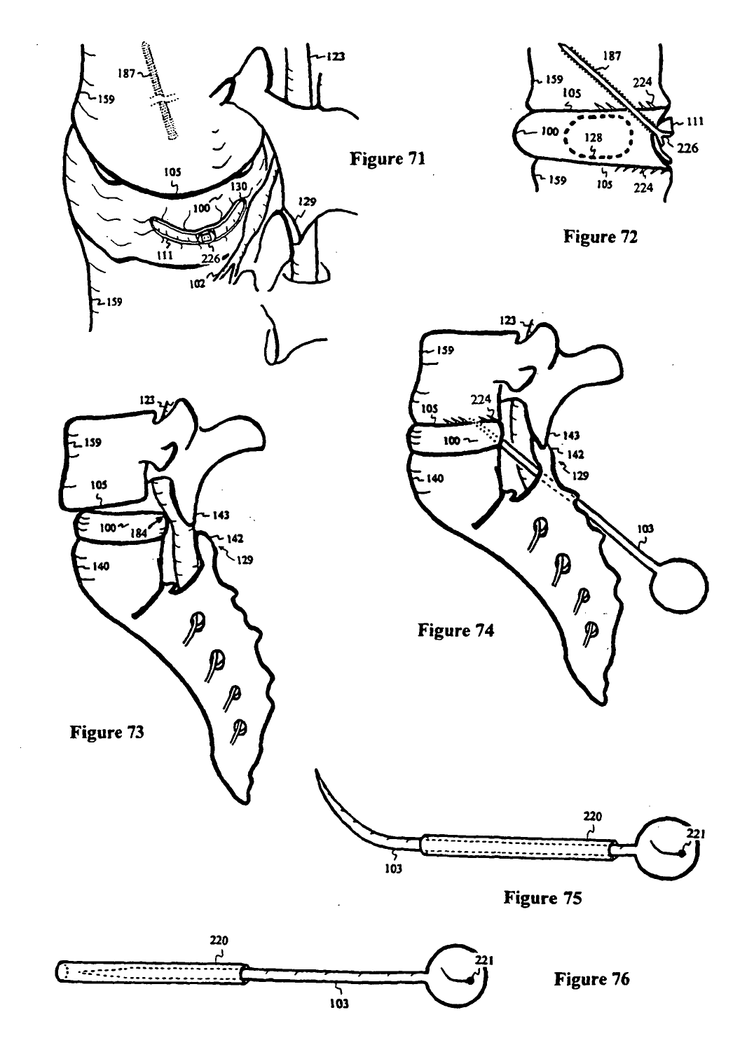

The compressor 111 can also be fastened through the outer layers of the disc 100, and/or with a bracket 139 fastened on the vertebral body 159, as shown in FIG. 66. The screw entry 217 can be made with a trocar 103, as shown in FIG. 67. To enhance annular reattachment and/or regeneration of the otherwise slow healing, avascularized annulus, bleeding sites 224 at the end-plate 105 are created by the trocar 103 through the bulging disc 100, as shown in FIG. 67. The entry of the trocar 103 depicted in FIG. 67 is slanted or angled upward, able to fit between the superior and inferior surfaces of the laminae, to prevent or minimize laminectomy. FIG. 68 shows a curved trocar 103 inflicting bleeding sites 224 in both superior and inferior end plates 105, through a posterior/lateral approach. A saddle-shaped compressor 111 is shown in FIG. 69 with a cylindrical annular contact surface 119, sloped surface 170, round contour tips 130, a screw hole 110 and a trough 223 or indentation to conceal the screw head 226 of a screw 187. FIG. 70 depicts penetration of the screw 187 through the outer portion of a protruded disc 100 and the end plate 105 into the vertebral body 159. FIG. 71 shows compression of the protruded disc 100 by the compressor 111 fastened by the screw 187 anchored in the vertebral body 159 to alleviate nerve 102 impingement 184 shown in FIG. 70. FIG. 72 shows a longitudinal view of a fastened disc 100 by the compressor/screw 111/187 with bleeding sites 224 inflicted on both end plates 105.

The strength of the fastened disc 100 may be greatly enhanced by healing initiated by the surgically inflicted bleeding sites 224. Ligament reattachment to bone is a good example. A biodegradable suture rated merely for 20 pounds is used to attach a torn ligament onto a surgically inflicted bleeding bone. Within two weeks, the tensile strength of the reattached ligament can reach 50 pounds; strength increases with time. In essence, the suture is merely used to maintain the position of the torn ligament; reattachment and healing occur naturally with the surgically inflicted bleeding bone. As the bulging annulus is compressed by the compressor 111 as shown in FIG. 72, adhesions form from oozing of the bleeding sites 224 between the end plate 105 and the compressed annulus. Tissue adhesion and the fastened compressor 111 work in conjunction to hold the bulging annulus in place, alleviate nerve 102 impingement 184 and allow time for the annulus to regenerate.

Similar to menisci in knees and articular cartilage in joints, the annulus has a limited capacity for healing and regeneration. For articular cartilage regeneration in the knee, an arthroscopic awl is used to create multiple holes on the articular cartilage surface, allowing blood and marrow elements to fill the defect, leading to formation of fibrocartilage. Patients have reported feeling significant improvement (Blevins F. T., et. al., Treatment of Articular Cartilage Defects in Athletes: An Analysis of Functional Outcome and Lesion Appearance, Orthopedics, July 21(7):761-7, 1998). No work has been done on end plate 105 puncturing to promote annular regeneration and adhesion. A qualitative in vitro investigation of adult human discs 100 showed that the end plates 105 are indeed partly permeable to solutes or nutrients. The permeation is associated with the presence of vascular contacts between the marrow spaces of the vertebral body 159 and the hyaline cartilage of the end plate 105. One-third of the central portion and only one-tenth of the peripheral zone of the end plates 105 are available for diffusion, exchanging nutrients and waste between the disc 100 and vertebral bodies 159 (S. Holm, et. al., Nutrition of the Intervertebral Disk, Clinical Orthopaedics and Related Research, 129, November-December: 101-14, 1977). It has been suggested that nutritional deficiencies could lead to disc 100 degeneration (Nachemson A., et. al., In vitro diffusion of dye through the end plates and the annulus fibrosus of human lumbar intervertebral disks, Acta Orthop. Scand., 41:589, 1970). It has also been suggested that annular regeneration is slow due to calcified hyaline cartilage at the end plate 105 in adults, which greatly hinders transportation of nutrients. End plate 105 punctures with an awl or trocar 103 could provide passages for nutrients, leading to the acceleration of annular regeneration. Furthermore, as the disc 100 undergoes rapid repair through the open channels created in the end plate 105, it is possible that fewer pain signals and/or shorter durations of them will be emitted from the degenerated annulus. Nerve 216 ingrowth into the disc 100 may decrease; the risks of future discogenic pain may decrease as well.

Spondylolisthesis is a condition in which a vertebral body 159 detaches and slips from a disc 100, usually the L5 and S1 disc 100, as shown in FIG. 73. The slippage usually occurs with some erosion on the facet joint 129, allowing the inferior articular process 143 of L5 to slip over the superior articular process 142 of S1, also shown in FIG. 73. Spondylolisthesis is normally surgically treated with lumbosacral fusion using instrumentation fastened by screws vulnerable to fatigue and breakage. Instead of using instrumentation to fuse the intervertebral segments, annular adhesion and regeneration may eliminate the need of instruments and hardware. After the spine with the affected vertebral body 159 is repositioned, bleeding sites 224 are created by the trocar 103 to initiate tissue adhesion between the end-plate 105 and the disc 100, as shown in FIG. 74. A period (2-4 weeks) of low back immobilization followed by passive motion is required for proper adhesion and adequate reattachment to take place.

A curved trocar 103 made with resilient material, such as nickel-titanium or spring tempered stainless steel, is housed in the lumen of a rigid sleeve 220, as shown in FIG. 75. The handle of the trocar 103 contains a label 221 indicating the direction of the curvature. The curved trocar 103 can be resiliently straightened within the sliding sleeve 220, as shown in FIG. 76. The curvature resumes when the sleeve 220 slides away from the curved section of the trocar 103. The sleeve/trocar 220/103 assembly is placed perpendicular to the disc 100. By pushing on the handle of the trocar 103, the trocar 103 pierces through the disc 100, resumes the unrestricted curvature and pierces into the end plate 105, as indicated in FIG. 77. The resiliently curved trocar 103 provides the surgeon greater latitude in terms of patient safety and surgically accessible locations to create bleeding sites 224 at the end plate 105.

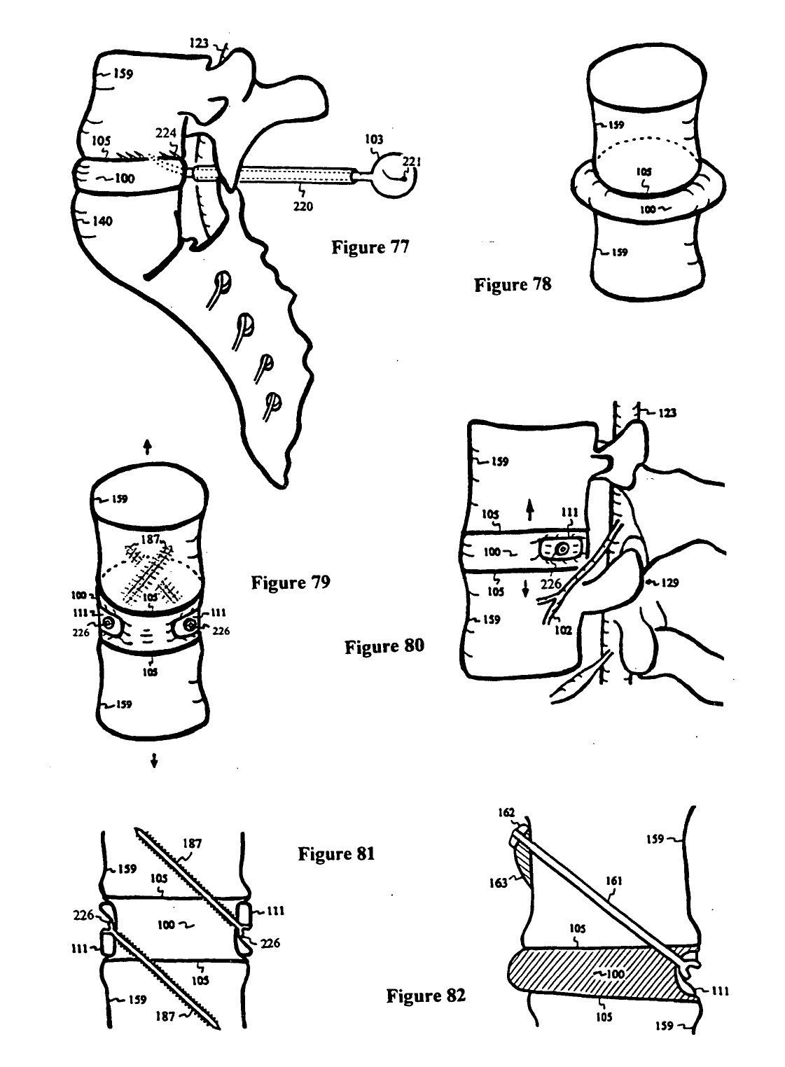

FIG. 78 depicts a flattened or bulging disc 100 sandwiched between vertebral bodies 159, a common cause of segmental instability and/or spinal stenosis. A pair of compressors/screws 111/187 is fastened through a portion of the disc 100, through the end plate 105 and into the vertebral body 159, as depicted in FIG. 79. The bulging or unstable sidewall of the disc 100 is compressed, supported, fortified, stiffened, restricted, tightened, pinched in and/or fastened by the compressors/screws 111/187 to minimize segmental instability.

A pair of compressors/screws 111/187 was used to fasten a cadaveric lumbar motion segment in similar fashion as FIG. 79. Motion analysis was done on the fastened cadaveric segment, showing significant increase in stability in flexion/extension and lateral bending motions. The disc height was also increased after disc 100 fastening with the compressors/screws 111/187. The result of the cadaveric study indicates potential for treating spinal stenosis by compressing, consolidating and tucking the bulging annulus back between the vertebral bodies 159 to build disc 100 thickness and intervertebral space and to alleviate nerve 102 impingement, as shown in FIG. 80. To prevent screws 187 from interfering with each other when multiple compressors 111 are used, screws 187 can be separately anchored into adjacent vertebral bodies 159, as shown in FIG. 81.

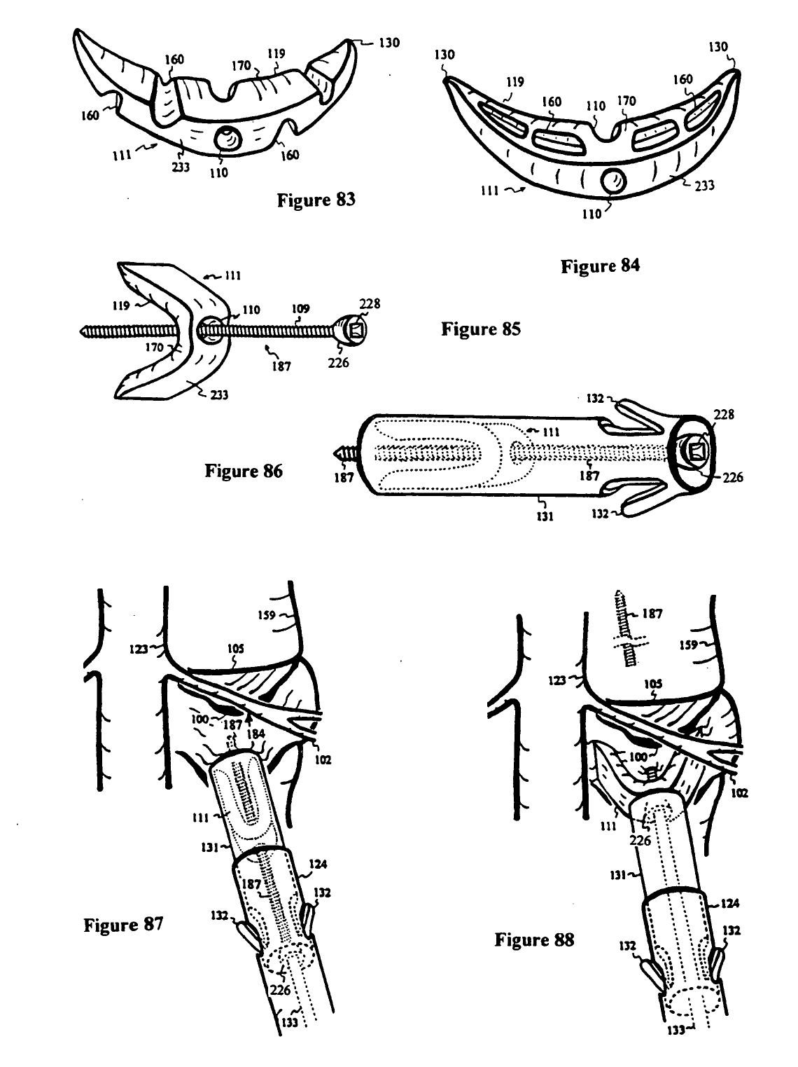

To minimize device migration, the compressor 100 can be fastened with a bolt 161 which penetrates obliquely through the vertebral body 159 and is fastened by a washer 163 and nut 162 assembly, as shown in FIG. 82. Promoting tissue ingrowth into the device can also minimize device migration. FIG. 83 depicts a compressor 111 with tissue ingrowth openings 160, channels or indentations to promote annular ingrowth and prevent migration of the compressor 111.

The compressor 111 shown in FIG. 84 also indicates multiple tissue ingrowth openings 160 penetrating through the thickness of the compressor 111. The large ingrowth openings 160 encourage annular ingrowth to prevent device migration with time. Different types of tissue ingrowth can be selected by varying the thickness of the compressor 111. The thick compressor 111 with large ingrowth openings 160 fastened adjacent to or over the end plates 105 may encourage bone ingrowth and promote segmental fusion without removing the disc 100. Existing spinal fusion procedure with discectomy often contributes to disc space narrowing, which may result in further nerve impingement. The segmental fusion induced by the bone ingrowth from upper and lower vertebral bodies 159 into the compressors 111 is accomplished after the distraction of the disc 100 with possible thickening of disc space. Osteoconductive material, such as bone growth factor collagen and/or hydroxyapatite, can be used to fill the tissue ingrowth openings 160. The surfaces of the compressor 111 can also be textured or made porous, similar to hip prostheses, to promote bone ingrowth.

For discs 100 at the thoracic or cervical region, rotational motion is also significant. FIG. 84 depicts a compressor 111 with tips 130 slightly curved outwardly to minimizing annular puncture during excessive or unforeseen rotations.

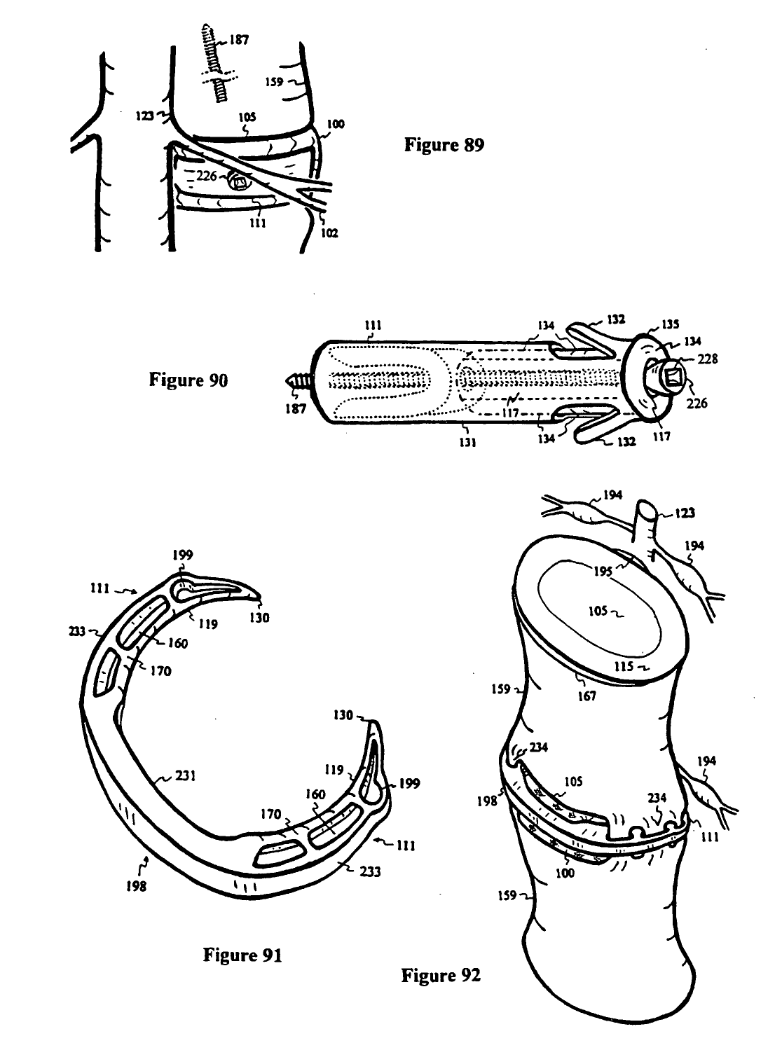

The compressor 111 can be made with a resilient or elastic material, such as nickel titanium, allowing up to 7% strain without losing shape memory. FIG. 85 depicts a compressor 111 in an open or predisposed position. The resilient compressors 111 can be folded or restricted in a tubular delivery capsule 131, as shown in FIG. 86, for endoscopic insertion. In the capsule 131, the resilient compressor 111 is in a delivery position. The delivery capsule 131 assembly holding the resilient compressor 111 and a screw 187 is fitted into a delivery device 124, secured by latches 132 and releasable by pinching, as shown in FIG. 87. The delivery device 124 is equipped with a drive 133 extending into the socket 228 opening of the screw 187. With a small diameter or cross section of the delivery capsule 131, it may be possible to reach the protruded disc 100 in the central zone by inserting the capsule 131 between laminae without laminotomy, as indicated in FIG. 87. The screw 187 is then advanced through the disc 100 into the end plate 105. As the screw head 226 contacts the compressor 111, the advancing screw 187 repels the restricted compressor 111 out of the capsule 131, as shown in FIG. 88. To keep the resilient compressor 111 from rotating with the screw 187, the cross section of the capsule 131 can be made non-circular. The repelled compressor 111 resumes the open position, spreading the legs of resilient compressors 111 on the protruded disc 100, anterior to the nerve 102. With further tightening of the screw 187 into the end-plate 105, the screw head 226 presses against the compressor 111, further spreading into a compressed position to fasten the previously bulging annulus, as shown in FIG. 89.

The resilient compressor 111, capsule 131 and screw 187 assembly is uniquely designed to accommodate the large moving range of the compressors 111 from the delivery position to the compressed position, a range even nickel-titanium alloy may not be able to provide. The uniqueness is in the open position, about half way between delivery and compressed positions. The magnitudes of the strain from the open to delivery position and from the open to compressed position are nearly equal but in opposite directions. In essence, the open or predisposed position is set at midway, making the large moving range of the compressor 111 possible, without shape memory loss.

To minimize swaying of the screw 187 during tightening, a stabilizer 134 is inserted in the capsule 131 to restrict the screw head 226 within a lumen 117 of the stabilizer 134, as shown in FIG. 90. The stabilizer 134 contains a lip 135 to prevent the stabilizer 134 from passing through the capsule 131. As the screw head 226 in the lumen 117 advances through the disc 100, lateral movement is greatly minimized during rotation of the socket drive 133.

FIG. 91 depicts a clamp/compressors 198/111 with large tissue ingrowth openings 160 to ensure annular ingrowth and prevent migration of the clamp/compressor 198/111. The widening mounts 199 can also be a portion of the ingrowth openings 160. The large ingrowth openings 160 may also allow bone ingrowth to promote spinal fusion 234 between upper and lower vertebral bodies 159, as shown in FIG. 92. The spinal fusion 234 induced by the compressors 111 can be further promoted by thick and porous compressors 111 bridging between two adjacent vertebral bodies 159, allowing the bone from adjacent vertebral bodies 159 to grow into the ingrowth openings 160 of the compressors 111. It is also possible to make the compressors 111 osteoconductive as hip and joint implants are, allowing bone from adjacent vertebral bodies 159 to embed and fuse with the compressors 111 and create segmental fusion 234. The uniqueness of this spinal fusion 234 is that it is accomplished with an intact and repaired disc 100 with the possibility of increased disc height induced by disc 100 compression. Similarly, compressors 111 with osteoconductive property, porous or large ingrowth openings 160 fastened with a bracket 139, bolt 161 or a screw 187 would provide bone ingrowth and spinal fusion 234.

A wide range of materials can be used to fabricate the compressor 111. Titanium, stainless steel, nickel-titanium alloy or other metallic material is preferred for strength and durability. To minimize tissue erosion, at least a portion of the compressor 111 can be made with biocompatible polymers, such as polyurethane, polypropylene, polyethylene, poly-ether-ether-ketone, acetal resin, polysulfone, polytetrafluoroethylene, polycarbonate, silicon, polyimide, ultra high molecular weight polyethylene or other. The compressor 111 can also be coated with lubricant, growth factor, nerve ingrowth inhibitor, nutrient, buffering agent, collagen, hydroxyapatite, analgesic, sealant for nucleus pulposus, blood clotting, antibiotic, radiopaque or echogenic agents. The casing 188 with pivotal peg 172, as shown in FIG. 21, can be made with stainless steel, titanium, nickel-titanium or a rigid polymer.

After the dysfunctional disc 100 has been repaired by the compressor 111, perhaps accelerated by the surgically inflicted bleeding sites 224, new annulus forms in a non-bulging position. Within months the strength of the repaired disc 100 may be mainly supported by the regenerated annulus cushioned between the vertebral bodies 159, rather than from the fastening strength of the compressor 111. Therefore, it may be possible to fabricate the compressor 111 and the supporting devices with biodegradable material, such as poly-lactate, poly-glycolic, polycaprolactone, trimethylene carbonate, combinations of these or other materials. A biodegradable device is particularly suitable for young patients to avoid device migration or other related complications in the distant future. All materials should be able to withstand sterilization by gamma, electron beam, steam, ETO, plasma or UV light to prevent infection.

Twenty to forty percent of patients undergoing laminectomy and/or discectomy procedures do not find pain relief. Due to the high invasiveness of present procedures, epidural scarring and vertebral instability are the most common and often lingering post-surgical complications. These tissue-removing procedures are not reversible. For many patients, the pain often returns in five years or less. In contrast, the proposed compressors 111 and methods repair the dysfunctional discs 100 without tissue removal, minimizing epidural scarring and strengthening the vertebral segment. Disc compression thickens the disc 100 and distracts the adjacent vertebral bodies to alleviate pain without removing tissues and weakening the spine. The proposed devices are retrievable, and the methods do not involve with tissue removal. Discectomy, laminectomy, foraminotomy, traditional spinal fusion or other conventional procedures can be used as a fall back procedure in the event of an unsuccessful outcome.

In summary, the compressors 111 on a clamp 198, a bracket 139, a bolt 161 (elastic or otherwise) or a screw 187 are used for (1) compressing a protrusion to alleviate impingement, (2) fortifying the annulus to stabilize a motion segment, (3) minimizing the inward/outward bulging to protect the disc 100 from progressive delaminations, (4) atrophying the nerve to treat discogenic pain, (5) correcting the curvature of spinal deformities, (6) elevating the disc space to treat spinal stenosis, (7) sealing the leakage of nucleus pulposus to treat herniated discs 100, and/or (8) promoting bony ingrowth to fuse the motion segment.

It is to be understood that the present invention is by no means limited to the particular constructions disclosed herein and/or shown in the drawings, but also includes any other modification, changes or equivalents within the scope of the claims. Many features have been listed with particular configurations, curvatures, options, and embodiments. The bracket 139 or the fusion plate in FIG. 50 can also be viewed as the extended stop 173 of the compressor 111. Any one or more of the features described may be added to or combined with any of the other embodiments or other standard devices to create alternate combinations and embodiments.

It should be clear to one skilled in the art that the current embodiments, materials, constructions, methods, tissues or incision sites are not the only uses for which the invention may be used. It has been foreseen that the elastic bolt 161, resiliently curved trocar 103 and/or resilient compressor 111 can be applied for other surgical and non-surgical purposes. Different materials, constructions, methods or designs for the compressors 111, brackets 139 or the delivery devices 124 can be substituted and used. Nothing in the preceding description should be taken to limit the scope of the present invention. The full scope of the invention is to be determined by the appended claims.

1. A disc space inserting device for inserting between adjacent vertebral bodies, said disc space inserting device comprising:
a superior plateau and an inferior plateau within a vertical plane extending therethrough, wherein said superior and inferior plateaus are adapted to be disposed between two adjacent vertebral bodies,
said superior plateau having a superior convex curvature within a superior horizontal plane, said superior convex curvature adjoining a generally perpendicular concave bracket,
said inferior plateau having an inferior convex curvature within an inferior horizontal plane, said inferior convex curvature also adjoining said generally perpendicular concave bracket, wherein said generally perpendicular concave bracket is sized and configured to partially fit around a vertebral body of the two adjacent vertebral bodies,
said superior and inferior convex curvatures are adapted to be disposed outside partially along the perimeter of the two adjacent vertebral bodies,
said superior plateau further comprising a superior inside edge extending from both ends of said superior convex curvature of said superior plateau within said superior horizontal plane, and said inferior plateau further comprising an inferior inside edge extending from both ends of said inferior convex curvature of said inferior plateau within said inferior horizontal plane, wherein said superior and inferior inside edges adjoin to form a disc-space-occupying protrusion adapted to be disposed between the two adjacent vertebral bodies,
said concave bracket further comprising at least one attachment hole, wherein said at least one attachment hole is sized and configured to fit at least one screw, thereby said disc space inserting device is fastened by said at least one screw in the vertebral body.

2. The disc space inserting device of claim 1, further comprising a generally perpendicular inferior concave bracket extending from said generally perpendicular concave bracket, wherein said generally perpendicular inferior concave bracket is sized and configured to partially fit around a second vertebral body of the two adjacent vertebral bodies,
said generally perpendicular inferior concave bracket further comprising at least one attachment opening, wherein said at least one attachment opening is sized and configured to fit at least one second-screw, thereby said disc space inserting device is fastened by said at least one second-screw in the second vertebral body.

3. The disc space inserting device of claim 1, wherein said superior plateau has an uneven surface.

4. The disc space inserting device of claim 1, wherein said inferior plateau has an uneven surface.

5. The disc space inserting device of claim 1, wherein said superior and inferior plateaus are nonparallel.

6. The disc space inserting device of claim 1, wherein said superior and inferior plateaus are generally parallel.

7. The disc space inserting device of claim 1, wherein said superior inside edge is tapered, thereby facilitating insertion of said superior plateau into and between said two adjacent vertebral bodies.

8. The disc space inserting device of claim 1, wherein said inferior inside edge is tapered, thereby facilitating insertion of said inferior plateau into and between said two adjacent vertebral bodies.

9. The disc space inserting device of claim 1, wherein said disc-space-occupying protrusion is curved.

10. The disc space inserting device of claim 1, wherein said at least one attachment hole is slanted, thereby said at least one screw fastening into the vertebral body is slanted.

11. The disc space inserting device of claim 2, wherein said at least one attachment opening is slanted, thereby said at least one second-screw fastening into the second vertebral body is slanted.

12. The disc space inserting device of claim 1, further comprising at least one indentation around said at least one attachment hole, thereby said at least one screw is concealed within said concave bracket.

13. The disc space inserting device of claim 2, further comprising at least one indentation around said at least one attachment opening, thereby said at least one second-screw is concealed within said inferior concave bracket.

14. The disc space inserting device of claim 1, further comprising tapered edges around said generally perpendicular concave bracket.

15. The disc space inserting device of claim 2, further comprising tapered edges around said generally perpendicular inferior concave bracket.

16. The disc space inserting device of claim 1, wherein said disc space inserting device is formed of a polymer.

17. The disc space inserting device of claim 1, wherein said disc space inserting device is formed of an alloy metal.

18. The disc space inserting device of claim 1, wherein said superior plateau and said inferior plateau having at least one tissue ingrowth opening.

19. A method of occupying intervertebral disc space, the method comprising the steps of:
(a) inserting horizontally oriented superior and inferior plateaus into a disc space;
(b) approximating a vertically oriented concave bracket adjoining said horizontally oriented superior and inferior plateaus to a vertebral body adjacent to the disc space;
(c) fastening at least one screw into the vertebral body through at least one hole in said concave bracket, thereby fastening the horizontally oriented superior and inferior plateaus in the disc space.

20. A method of occupying intervertebral disc space, the method comprising the steps of:
(a) inserting horizontally oriented superior and inferior plateaus into a disc space;
(b) approximating a vertically oriented concave bracket adjoining said horizontally oriented superior and inferior plateaus to a superior and inferior vertebral bodies adjacent to the disc space;
(c) fastening at least one screw into the superior vertebral body through at least one hole in a superior portion of said vertically oriented concave bracket;
(d) fastening at least one additional screw into the inferior vertebral body through at least one additional hole in an inferior portion of said vertically oriented concave bracket, thereby fastening the horizontally oriented superior and inferior plateaus in the disc space.

21. The method of occupying intervertebral disc space of claim 20, wherein the method is used to treat spinal stenosis.

22. The method of occupying intervertebral disc space of claim 20, wherein the method is used to treat kyphosis.

23. The method of occupying intervertebral disc space of claim 20, wherein the method is used to treat scoliosis.

24. The method of claim 20, wherein said at least one screw is attached through an end plate of the vertebral body.
